(19) United States
(12) Patent Application Publication
(10) Pub. No.: US 2025/0135454 A1
(43) Pub. Date: May 1, 2025

(54) CLOSED MICROFLUIDIC CARTRIDGE FOR EVALUATING MULTIPLE, ISOLATED PORTIONS OF A FLUID SAMPLE

(71) Applicant: Coagulo Medical Technologies, Inc., Auburndale, MA (US)

(72) Inventors: Galit H. Frydman, Boston, MA (US); Leonid Silverman, Cranston, RI (US)

(21) Appl. No.: 18/682,932

(22) PCT Filed: Aug. 9, 2022
(86) PCT No.: PCT/US2022/039851

§ 371 Date: Feb. 11, 2024

Related U.S. Application Data
(60) Provisional application No. 63/232,453, filed on Aug. 12, 2021.

(51) Int. Cl. B01L 3/00

(52) U.S. Cl. CPC B01L 3/502715; B01L 2200/0605; B01L 2200/16; B01L 2300/0645; B01L 2300/0663; B01L 2300/0864

(57) ABSTRACT

The present invention relates to microfluidic cartridges that operate as closed systems. A microfluidic cartridge as described herein allows for the independent evaluation of multiple, isolated portions of a fluid sample (such as, e.g., a blood sample). In certain embodiments, each isolated portion has a predetermined volume. The present invention also relates to methods for simultaneously evaluating multiple, isolated portions of a fluid sample using a single cartridge.

### FIELD OF THE INVENTION

The present invention generally relates to devices, systems, and methods for evaluating multiple, isolated portions of a fluid sample. The devices, systems, and methods described herein can be used for various applications, including, for example, in medical diagnostics for evaluating biological fluid samples, such as for assessing coagulation in a blood sample.

### BACKGROUND OF THE INVENTION

The use of microfluidics for diagnostics and other applications is becoming increasingly common. Most current microfluidic cartridges are open systems, where there is open communication between the internal contents of the cartridge and the external environment or a portion of an automated reader, such as a syringe. Some of these systems are stated to minimize the chance of contamination to or from the outside environment or reader parts, such as through the use of specialized filters located near the outlet or inlet ports. However, none of these systems eliminates the risk of contamination. Contamination can be dangerous, especially when working with biological or environmental samples that may be harmful upon aerosolization. Additionally, an open system with filters is not suitable for evaluating gas-phase fluid samples, as the gas in such samples may escape through the pores of the filters. Similarly, for systems that use valves to control fluid flow, such valves may be “liquid-tight” but not “air-tight,” and thus air may escape through such valves. Other cartridges may include a waste or other chamber that is open (e.g., a vented waste chamber) and not closed, in order to reduce the internal pressure in the cartridge. See, e.g., Miyazaki et al., Processes 8:1360 (2020); Ahrberg et al., Lab Chip 16:3866-84 (2016); Al-Faqueri et al., Sensors 15:4658-76 (2015). The waste chamber in such cartridges serves as an outlet and opening to the external environment.

There is a critical need in the art for devices that are closed systems once a sample has been inserted (e.g., to contain dangerous pathogens or other hazardous agents within a single-use test cartridge). There also is a need in the art for devices, systems, and methods for the evaluation of multiple, isolated portions of a fluid sample, such that multiple portions of a single fluid sample can be evaluated independently and simultaneously using a single device. There is a particular need for devices of decreased cost and/or decreased complexity that permit splitting a fluid sample into multiple, isolated portions using stationary features within the device (e.g., using the geometry of microfluidic channels) and that permit exposing each portion to one or more reagents independently of the other portions (e.g., to avoid cross-talk between portions, and to avoid the propagation of any chemical or physical reaction or signal from one portion of the sample to another portion). The present invention addresses these needs.

### SUMMARY OF THE INVENTION

The present invention provides devices, systems, and methods for evaluating a fluid sample. The devices can be used, for example, for the in vitro evaluation of a blood sample, to assess a subject's coagulation status and/or phenotype. The devices also can be used to detect one or more analytes in a fluid sample. An example device comprises a microfluidic cartridge having a plurality of channels, each channel of the plurality of channels configured to receive a portion of the sample through a common inlet connected to a first end (which may be referred to as the upstream end) of the plurality of channels, and each channel of the plurality of channels leading to a downstream chamber (or series of chambers, etc.) in the channel. A downstream chamber may be a metering chamber or a waste chamber. Downstream of each of the metering chambers may be another chamber (or series of chambers, etc.) in which some measurement is taken and/or some other analysis occurs. A chamber (or well, etc.) where analysis occurs is referred to herein as a testing chamber, sensing chamber, measurement chamber, testing well, sensing well, etc. Downstream of each of the testing chambers is an air spring or other closed chamber. The aforementioned chambers may be connected by channel paths of various shapes and lengths, as further described herein.

Embodiments of the present invention also provide a device for evaluating a fluid sample, the device comprising: a microfluidic cartridge having a plurality of channels, which cartridge accepts a fluid sample through a common inlet connected to a first end of the plurality of channels (such that each of the channels connects to the inlet), and wherein the plurality of channels comprises at least three channels, such that the fluid sample is split into at least three channels. In some embodiments, at least two of the channels each has a predetermined target volume (e.g., a metered volume), and in further embodiments, the predetermined target volume is dictated by the geometry of each such channel. In certain embodiments, each of the at least two channels may comprise a metering chamber, such that the fluid sample that is in each of the at least two channels fills one or more metering chambers. In some aspects, the fill of the metering chamber in each of such channels is based on pressure differentials caused by the size and shape of each of the channels, as described herein. At least one of the channels of the plurality of channels comprises a waste chamber at its downstream end.

In embodiments of the devices, systems, and methods of the present invention, the cartridge may be a microfluidic cartridge, chip, or other device, and the plurality of channels may be microfluidic channels. In some embodiments, the methods can be performed using a device requiring less than about 1 mL, less than about 500 μL, less than about 100 μL, or less than about 50 μL, or about 5 μL or less, of a fluid sample (e.g., in some embodiments, about one drop of fresh whole blood or whole blood anticoagulated with sodium citrate, is sufficient). In some embodiments, channel width, channel height, or both, may be less than 10 mm, less than 6 mm, or less than 2 mm. In some embodiments, a single channel may have varying dimensions (e.g., width, height, radius, etc.) along a length of the channel (for example, certain regions along a channel's path may be wider or have a greater radius than other regions); in addition, one or more of the plurality of channels may have different dimensions than the other channels. The channels (and the chambers and other regions along the length of each channel) may be of various shapes, such as, for example, equilateral polygons (e.g., hexagon), asymmetrical polygons (e.g., a six-sided polygon with various side lengths), or straight channels (e.g., a rectangular or cylindrical channel). Further, for any geometry used, the corners and edges where two faces meet may be rounded (e.g., to promote fluid flow through the channel). In certain embodiments, it may be desirable to use a variety of different channel geometries. Depending on the embodiment, one or more of the channels of the plurality of channels (e.g., all of the channels) may have the same volume, or the volume may vary from channel to channel. Similarly, in embodiments wherein one or more of the plurality of channels comprises multiple functional regions (e.g., a metering chamber, a reagent chamber, a mixing chamber, a sensing chamber, etc.) connected in series, all such regions may have the same volume, or volume may vary from region to region (e.g., in a preferred embodiment, a metering chamber may have a volume of about 3 μL, a reagent chamber may have a volume of about 1 μL, and a sensing chamber may have a volume of about 2 μL). In addition, the material(s) that form the channels (and thus the material(s) that line the interior surface of the channels, for example) may be the same across one or more channels or may differ from channel to channel. The channels' relative locations to one another also may vary, depending on the embodiment. For example, the channels all may be formed within the same substrate and located on the same plane; the channels all may be formed within the same substrate, but may be situated on different (e.g., parallel) planes; the channels may be formed in multiple substrates (e.g., in some embodiments, by layering materials) and located on a single plane; or the channels may be formed in multiple substrates and located on different planes (e.g., as in a multi-planar cartridge with some channels formed within one plane of a substrate and other channels located on another plane, and wherein at least one of the walls of the channels on one or more planes is comprised of a second substrate, such as a layered material, e.g., pressure sensitive adhesive).

In various embodiments of the devices, systems, and methods of the present invention, the cartridge may contain sensors (e.g., electrode sensors). In some embodiments, the part of the device that houses the sensors may also form one or more of the walls of one or more of the channels. Alternatively, in certain embodiments, one or more of the walls of one or more of the channels may be made from the sensor itself—e.g., as in a multi-layer PCB (printed circuit board) with one or more exposed electrode tracings. In embodiments in which a multi-layer PCB with exposed electrode tracings is used, an exposed electrode tracing may act as a sensor, or it may serve other purposes, such as, e.g., serving as an integrated heating or cooling unit. Sensors may be exposed electrical tracings (e.g., a pair of electrodes, interdigitated electrodes, etc., for measuring impedance) or may be other types of sensors attached to a PCB (e.g., a photodiode, camera lens, spectrophotometer, photonic sensor, piezo sensor, etc.). In some embodiments where a PCB is used, the PCB may include a stimulant (e.g., a light source, etc.) and a receiver (e.g., a photodiode, etc.).

In some embodiments, the cartridge may include multiple sensors, where the sensors are of the same type (e.g., electrode sensors that measure an electrical property, such as impedance) or of various types. For example, a cartridge may comprise one type of sensor on one side and a different type of sensor on the other side; for any sensor, the sensor may be part of or attached to a PCB, as discussed above. In certain embodiments, a multi-layer cartridge may comprise a PCB as a layer, wherein the PCB comprises a sensor or sensors, and the cartridge may further comprise additional sensors within or on one or more other layers. Sensor layers can be made from multiple individual or connected PCBs, such as a PCB that is a bottom layer of a cartridge and that communicates with a PCB that is a top layer of a cartridge; such communication may be achieved via a light source and photodiode, etc., where the PCBs are positioned such that they measure activity and/or characteristics (e.g., by intercepting signals) of a portion of a fluid sample in a channel. In some embodiments, two PCBs can align with each other to form a channel, while each PCB may take different measurements (e.g., one PCB comprises sensors that measure electrical impedance, and a second PCB comprises sensors that take optical measurements, such as for machine learning, artificial intelligence, etc.). In certain aspects, two PCBs, each PCB comprising sensors, can be independent of each other and measure different properties within the same channel (at the same location in the channel or at different regions of the same channel), or they may measure different properties in different channels and/or within different parts of the cartridge (e.g., through the use of two PCBs, each PCB comprising sensors, where electrical impedance can be measured in one channel on one side of the cartridge, and optical sensing can be performed in another channel on the other side of the cartridge).

In some embodiments, a PCB may comprise electrodes, for example, as sensors to measure a property of the fluid sample and perform testing, or for sensing the position of the fluid (e.g., as fluidic position sensors).

In some embodiments, the cartridges as described herein may comprise fluidic position sensors. Fluidic position sensors may be based on electrical impedance using electrodes, for example (and as mentioned above), or may be made of another component attached to the PCB. In various embodiments, fluidic position sensors may provide active feedback to the external component actuating the cartridge, e.g., to ensure fluidic movement within the cartridge and/or for precise control of fluidic movement. Such a feature, depicted, for example, in the embodiments illustrated in the figures, is particularly advantageous, as it allows a single cartridge design to be suitable for testing various types of fluid samples with varying viscosities (e.g., blood samples of varying hematocrits, urine and saliva, etc.). Accordingly, in some embodiments, a fluidic position sensor may provide feedback to an external control system; such sensor and feedback may be used to detect a fluid's position within a cartridge and make appropriate adjustments to fluid flow (in certain embodiments, e.g., to confirm that a minimum required fill has been achieved) or to control fluidic movement (in various embodiments, e.g., by applying a particular amount of pressure in response to input received from the fluidic position sensor). For example, in some embodiments, a fluidic position sensor (which may be a pair of electrode sensors or may be another sensor that measures light, ultrasound, radiofrequency, absorbance, etc.) is used to detect the fill volume of a metering chamber, and once the fill volume of the metering chamber has reached a specific level, the speed or pressure applied to the actuator is modified (e.g., reduced) such that the remainder of the fluid sample that has not yet been split into a channel moves to a channel comprising a waste chamber instead of to a channel comprising a metering chamber.

In addition, in some embodiments where a microfluidic cartridge is used to evaluate a blood sample, the microfluidic cartridge may comprise sensors that measure hematocrit in the sample. Hematocrit information can be used to assess the sample's viscosity. Sensors that measure hematocrit and provide such hematocrit information to an external control system can be used to control and adjust fluidic movement via sensor feedback.

In certain embodiments, it may be beneficial to select specific features and/or materials to optimize testing conditions in a particular region of the cartridge. For example, certain layering materials may be selected (e.g., glass, either for a whole layer or to make optical windows, to facilitate measurement using optical sensors), or certain coatings may be applied (e.g., to alter surface topology), and/or certain treatments may be used (e.g., treating a region of the cartridge with a chemical, such as to make a surface hydrophilic or hydrophobic, or with a biological material).

Similarly, the material(s) of the cartridge body and/or cartridge layers may be selected to facilitate the fluidic dynamics that are best suited for the type of testing to be conducted in the cartridge (e.g., a plastic, such as polycarbonate, is more hydrophobic than glass and may be better suited than glass for certain types of analyses). The materials used may affect fluidic movement and the pressure within the system, similar to how viscosity of the fluid or the location of the fluid within the cartridge (e.g., whether the fluid is in the middle of a large chamber or is in a comparatively narrow resistor) can affect fluidic movement. For example, a more hydrophobic material lining the surface of the channels can help reduce capillary action and improve accurate metering, especially when the system is under sufficiently low pressure such that capillary action is possible. In addition, in some regions of the cartridge it may be desirable to line the surface of a channel with a material that facilitates sacrificial fluid retention, as described further herein.

The present invention also provides microfluidic cartridges that are useful as single-use, disposable cartridges. In other embodiments, a cartridge may be a reusable cartridge and may comprise features that facilitate cleaning the cartridge prior to reuse. As discussed above, the cartridges described herein may be made of a single material or of multiple materials.

In some embodiments, a plurality of channels is formed in a substrate, which can be made of one or more of various materials (e.g., acrylic, polycarbonate, liquid crystal polymer, etc.), and by any one or any combination of methods (e.g., injection molding, machining, laser etching, layering, etc.). In some embodiments, the cartridge comprises a primary substrate and one or more layers. For example, in certain embodiments, a plurality of flow paths may be formed in the primary substrate such that, in the substrate itself, the plurality of flow paths is open on one side (for instance, on the top side of the flow paths), and one or more cover layers may be attached to the primary substrate to fully enclose the flow paths molded in the substrate and form channels. While various embodiments are discussed with reference to “channels,” other terms (e.g., lanes, compartments, partitions, etc.) can describe spaces that are physically separated from each other and that, in some embodiments, have the same geometry as each other.

In certain embodiments of the devices, systems, and methods of the present invention, a cartridge has a common inlet connected to the first end of a plurality of channels. In some embodiments, the common inlet is the cartridge's only opening. In other embodiments, a cartridge may have one or more other openings, in addition to a common inlet. For example, a cartridge may have a common inlet and a vent port. Regardless of how many openings a cartridge may have, it is an essential feature of such embodiments that each opening be closed after sample input and prior to effecting movement of the fluid sample through the plurality of channels in the cartridge. For example, a vent port (if present) may comprise a porous membrane that swells, such that its pores close, when it is wetted by a fluid sample.

In some embodiments, the cartridge may have no internal moving parts. For instance, a cartridge may comprise a bellows, which can be actuated by an external component so as to push the volume of air contained in the bellows into the cartridge, as a way to control internal pressure and hence fluidic movement.

In some aspects, the cartridge may have a single moving part, which pressurizes the system and moves the fluid sample through a plurality of channels. A cartridge may include, for example, an integrated syringe with a plunger; in such embodiments, an external actuator may move the plunger to control pressure in the system. In all of these embodiments, the external component can selectively pressurize or de-pressurize the system in order to move the fluid forward or backward (e.g., for purposes such as mixing fluid, to move fluid to a particular position within a testing chamber, etc.).

In embodiments of the devices, systems, and methods of the present invention, the geometry of each of the plurality of channels may incorporate air springs and resistive regions.

In various embodiments of the devices, systems, and methods of the present invention, the cartridge may lack active control elements (other than, e.g., an actuator as described herein), valves, vents, and hydrophobic membranes.

In certain embodiments, internal (i.e., contained within the cartridge) control elements may be used to allow the closing of the system; such elements include, for example, a porous membrane, which swells so that its pores close upon contact with the fluid sample.

In embodiments of the devices, systems, and methods of the present invention, the cartridge may use forward and backward movement of the fluid sample (such as for mixing of the fluid sample with a reagent). In other embodiments, fluid flow may be unidirectional.

A fluid or fluid sample may include, without limitation, a sample of bodily fluid (e.g., blood, plasma, saliva, urine, cerebrospinal fluid (CSF), peritoneal fluid, thoracic fluid (including pleural fluid), or pericardial fluid), a sample of bodily gases (e.g., a sample from exhalation), chemical and/or environmental samples (including, without limitation, a sample of a chemical in liquid or gaseous form), a sample of water, or a sample of air.

As used herein, unless described otherwise, a “blood sample” refers to a whole blood sample or a plasma sample. The term plasma includes both platelet-rich-plasma (PRP) and platelet-poor-plasma (PPP). In any of the devices, systems, and methods described herein, the blood sample can be a whole blood sample or a plasma sample. Using whole blood can be particularly useful for certain applications, such as those implemented at the bedside of a patient.

In some embodiments, the fluid sample is a gas and the medium used to pressurize the system and move the sample downstream through channels can be another gas or air or, in some embodiments, may be a liquid. The medium used to pressurize the system may be selected so as to minimize reactivity with the sample and permeability to the sample. In embodiments where a medium other than air is used, there may be a container (e.g. a pouch, etc.) that holds this medium and that releases the medium upon the start of the cartridge test or with the start of the actuator (e.g., with the start of actuation of the bellows or syringe pump), such as with a perforating needle or other component.

The term “chamber” and variations thereof are used herein synonymously with the term “well” and variations thereof and are non-limiting terms. Such chambers are located along the paths of the microchannels. In some embodiments, a chamber may refer to a particular location within a channel, even if the channel's geometry at that particular location does not change and is the same as the channel's geometry before and after the chamber. In other embodiments, the channel's geometry may change at a chamber, such that, for example, the channel's volume increases at the location of a chamber. In addition, while various embodiments are discussed with reference to “measurement chambers,” “sensing chambers,” or “testing chambers,” other terms (e.g., testing wells, sensing wells, etc.) can also describe regions of a channel (or other areas of a cartridge) where some kind of measurement or analysis takes place.

The term “closed system” and variations thereof generally refer to a cartridge that is a dead-end system. A closed system uses pressure gradients to cause back pressure in specific places at various points in time to control fluid flow.

As used herein, unless specified otherwise, references to numbered features in the figures that include a letter (e.g., 140a) refer only to that specific alphanumerical feature; references to features that do not include a letter (e.g., 140) are intended to refer to all such numbered features (e.g., 140a, 140b, 140c, etc.).

### BRIEF DESCRIPTION OF THE DRAWINGS

FIG. 1 depicts the operational principle underlying the closed cartridge air spring design.

FIG. 2 extends the operational principle depicted in FIG. 1 to a multi-channel microfluidic cartridge, wherein the channels are connected to a common inlet at their upstream ends.

FIG. 3 models the time-dependent fluidic flow of a Hagen-Poiseuille system like the closed microfluidic air spring cartridge.

FIGS. 4A-4E depict fluidic flow at various, progressing states of cartridge pressurization. FIG. 4A depicts fluid flow at state 0. FIG. 4B depicts fluid flow at state 1.

FIG. 4C depicts fluid flow at state 2. FIG. 4D depicts fluid flow at state 3. FIG. 4E depicts fluid flow at state 4.

FIG. 5A and FIG. 5B each depict an embodiment of an air spring cartridge system.

FIG. 6A depicts an embodiment of an air spring cartridge system that has nine testing chambers and three waste chambers.

FIG. 6B depicts part of a channel (such as channel a of the embodiment depicted in FIG. 6A) that includes additional paths (e.g., additional chambers, or a series of additional chambers) and/or resistive features between the metering chamber (130) and the testing chamber (140).

FIG. 7 depicts the system corresponding to some of the cartridge embodiments described herein (e.g., in FIGS. 10A, 10B, and 11).

FIG. 8 shows how certain features in a single microfluidic channel (e.g., a single channel of the cartridge shown in FIGS. 10A-10B) are situated within a multi-layer microfluidic cartridge.

FIG. 9 shows an embodiment in which each of nine channels of a cartridge comprises multiple electrode pairs (each electrode pair is shown as a pair of dots). In certain channels, two electrode pairs are part of a feedback mechanism that is used to control actuation of the active element controlling fluid flow. In addition, another electrode pair (shown as the pair of dots in the center of the nine channels) is located in or near the common channel where the fluid sample resides before it is split into multiple channels; this electrode pair may serve as a sensor, and also may be used as part of the feedback mechanism used to control fluid flow.

FIG. 10A provides a top view and a bottom view of the multi-layer microfluidic cartridge employing the system depicted schematically in FIG. 7.

FIG. 10B shows various layers of the same cartridge embodiment depicted in FIGS. 8 and 10A.

FIG. 11 shows a top view of an embodiment of the microfluidic cartridge employing the system depicted schematically in FIG. 7.

FIGS. 12A-12B show embodiments of a cartridge in use, where a fluid sample has been inserted. FIG. 12A provides images showing a cartridge in use where the fluid sample is water that has been mixed with dye to facilitate imaging. FIG. 12B provides images showing a cartridge in use where the fluid sample is citrated whole blood.

FIGS. 13A-13F illustrate reagent mixing and demonstrate how the separated portions of a fluid sample remain isolated from each other as the portions flow through their respective channels in a cartridge. Dye of different colors was spotted into chambers, with each chamber receiving one or two colors (red, yellow, or red and yellow) (FIG. 13A), and allowed to dry (FIG. 13B). As shown in FIG. 13C, a water fluid sample was introduced into and pushed through an assembled cartridge, and there is complete separation of the fluid sample portions and no mixing of color between the channels. FIG. 13D shows how the dry-spotted dye(s) mix with the aliquots of the fluid sample in the mixing chambers, with uniform distribution of the dye(s) within the fluid sample in the mixing chambers. FIGS. 13E and 13F show images when the water fluid aliquots (after mixing with the dry-spotted red and/or yellow dye) have entered into the testing chambers (which had been dry-spotted with blue dye); in the testing chambers the blue dye does not uniformly disperse into and mix with the fluid sample, demonstrating that in this cartridge embodiment the mixing illustrated in FIG. 13D is necessary to achieve full mixing of the sample with dry-spotted dye(s).

FIG. 14 shows sacrificial retention of a fluid sample in a closed cartridge system; such sacrificial fluid retention can be used to accommodate evaporation of a fluid sample in certain embodiments.

### DETAILED DESCRIPTION OF THE INVENTION

The features and advantages of the present invention may be more readily understood by those of ordinary skill in the art upon reading the following detailed description. It is to be appreciated that certain features of the invention that are described above and below in the context of separate embodiments also may be combined to form a single embodiment. Conversely, various features of the invention that are described in the context of a single embodiment for reasons of brevity may also be combined so as to form sub-combinations thereof. In addition, the drawings and specific embodiments of the invention described herein are illustrated by way of example, it being expressly understood that the description and drawings are only for the purpose of illustration and that the specific embodiments are not intended to define the limits of the present invention.

The present invention provides a self-contained microfluidic cartridge for use in analyzing a fluid sample. A key feature of the disclosed embodiments is that they allow for independent evaluation of multiple, isolated portions of a fluid sample using a single cartridge. More specifically, the microfluidic cartridges described herein combine a parallelized air spring design with stationary features to control flow of a fluid sample inserted into the cartridge; such cartridges provide for the control of movement of the fluid sample through a closed system using only a single active element external to the cartridge (e.g., an actuator that operates a syringe, plunger, bellows, pump, etc.). The external active element of the cartridge pushes air and the fluid sample through a closed microfluidic cartridge, without the need for any additional valves or other control elements.

As discussed further below, there are two compartments of air in the closed cartridge systems described herein: one compartment of air is upstream of the fluid sample and the other compartment of air is downstream of the fluid sample. The upstream air is used to push the fluid sample portions and the air that is downstream of these portions, resulting in movement of the fluid sample downstream through the channels. This process results in compression and increased pressure, as the volumes holding the upstream air, fluid sample, and downstream air are made smaller.

The microfluidic cartridges described herein are dead-end systems (such that their flow paths are closed, with no openings such as vents, etc.) that rely on pressure gradients and back pressure to control fluid flow to and through the desired microfluidic channels (which may comprise one or more chambers) at various points in time. In some embodiments, the devices, systems, and methods described herein achieve these pressure gradients and back pressure using an air reservoir, air springs, and metering chambers. In some aspects, the pressure gradients and back pressure ensure unidirectional fluid flow through the microfluidic cartridge. In addition, some embodiments may also use resistors (e.g., a tesla valve, a meandering fluid flow path, etc.) to control and/or prevent backflow of a fluid sample.

The devices, systems, and methods described herein provide a number of benefits over existing microfluidic cartridges in that they provide for the following features:
a. each portion of the fluid sample can be metered to a known volume (referred to herein as an aliquot volume or metered volume), such that the volume of fluid sample contained in each of the channels, and in each chamber of a channel (e.g., a reagent chamber, a testing chamber) is of a known volume;
b. each portion of the fluid sample contained in each of the channels (and thus in each chamber of a channel (e.g., testing chambers as well as waste chambers)) is isolated from all other portions of the fluid sample that are contained in the other channels (and in any chambers along those other channels); and
c. the cartridge operates as a closed system and its contents are not exposed to the outside environment.

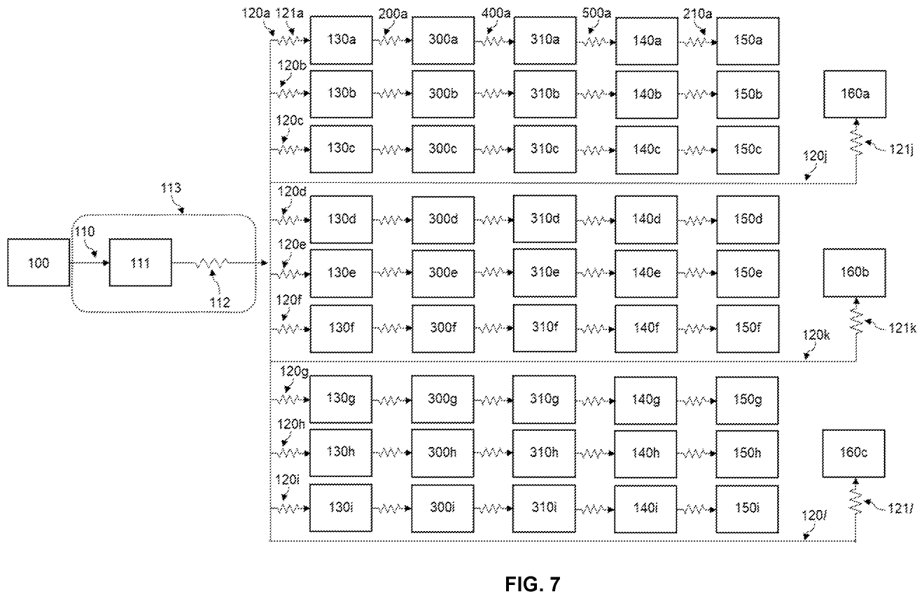

In some embodiments, such as embodiments where it is desirable to expose each portion of a fluid sample to a different condition (for example, to expose the portions to different reagents, or to different concentrations of a reagent or reagents), it is imperative to ensure that each portion is of a known volume (e.g., such that the concentration of an added reagent can be known and controlled). Thus, the cartridges described herein provide for metering portions of a fluid sample to achieve portions of known volumes (a portion of the fluid sample, once metered to a target volume, is called an aliquot). The target volumes (which are also called metered volumes) across the channels may be the same for all of the channels or may differ across channels. In addition to the portions of the fluid sample that are metered, one or more other portions of the fluid sample may not be metered and may be received into a channel comprising a waste chamber (for example, channels 120j, 120k, and 120/in FIG. 7). It is also critical to ensure that a chemical or physical reaction in one of the channels (e.g., in one of the channel's chambers) does not affect any chemical or physical reaction that may occur in any of the other channels (e.g., in chambers of the other channels).

In some embodiments, particularly embodiments where a hazardous and/or infectious fluid sample is being analyzed, it is desirable to have a closed system to contain the sample within the cartridge and ensure safety for the persons inserting the sample and/or running the analysis, as well as to allow for the safe disposal of the cartridge (which contains the fluid sample) after analysis and use of the cartridge.

Embodiments of the present invention provide microfluidic cartridges that operate as closed systems that contain a volume of fluid (which can be air or liquid) that is displaced in a controlled fashion to control the movement of an inserted fluid sample. The closed cartridges as described herein may rely on a single actuator to control fluid sample movement through the cartridge's channels, though other mechanisms of controlling movement of the fluid sample may be employed. For cartridges that rely on a single, closed actuator (e.g., plunger, bellows, etc.), without requiring other active control elements or valves, vents, hydrophobic membranes, etc., such cartridges can be made at a lower cost compared to other devices that require additional elements for controlling fluid sample insertion and flow. This cost-savings is of great benefit, for instance, for point-of-care or at-home analyses (e.g., point-of-care diagnostics), or in cases where tests are likely to be used in the field (e.g., water quality testing), in austere conditions (e.g., the battlefield, zero gravity, etc.), or at another point-of-need (e.g., infectious disease testing in remote locations abroad). Using a single, closed actuator also may facilitate assembly and promote manufacturability of the cartridges, as fewer parts are required for actuation of the cartridge. Similarly, using a single, closed actuator may increase the robustness of the cartridges, particularly when such cartridges are used as diagnostic instruments (e.g., the cartridges may have fewer moving and/or breakable parts).

In some embodiments, using a cartridge with a closed system may also obviate the need for complex fluid management (e.g., post-analysis procedures, such as washing) external to the cartridge, for example, in the analyzer that obtains measurements from the cartridge.

### Principles of Operation

The present invention generally relates to a closed air spring microfluidic cartridge that is useful for analyzing a fluid sample, for example for evaluating physical and/or chemical characteristics of a fluid sample.

Operation of the closed microfluidic cartridge can be explained by reference to the ideal gas law, which describes the behavior of a gas with respect to pressure (P), volume (V), temperature (T), and the amount (n) of gas, such that:

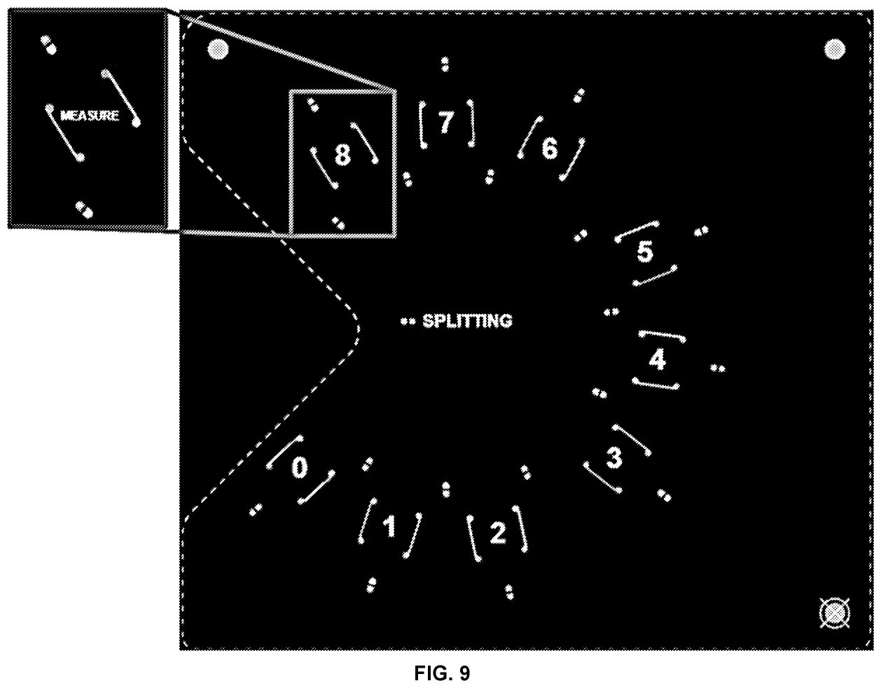

PV=nRT

where R is the ideal gas constant. For a closed microfluidic cartridge to function properly, distal air inside the cartridge must remain unvented during the testing process (in other words, the distal air (which is the air that becomes trapped downstream of the fluid sample, upon insertion of the fluid sample) must not be released from the closed cartridge during use). In some embodiments, where n, T, and R are kept constant throughout the testing process, there will be a direct and proportional relationship between P and V at any moment in time throughout the process. In other embodiments where Tis not kept constant, the system provides feedback to the actuator—e.g., via electrode sensors positioned at various locations in one or more of the channels, where such sensors determine the amount of fluid within the channels (for example, within particular chambers of the channels)—and through a feedback mechanism with the actuator, the actuator can then compensate for the pressure change within the system (e.g., by pushing more or less, and/or by pushing faster or slower). In some embodiments, the viscosity of the fluid sample also may play a role in pressure changes and the system's feedback to the actuator. The cartridge's feedback loop (see, e.g., FIG. 9) thus allows the system to accommodate such variables.

To illustrate the operation of the air spring system, the following discussion focuses on fluid movement through a single channel. In such example of a single channel, a fluid sample is introduced into a microfluidic cartridge at the first end of a microfluidic channel, and the fluid sample travels through the channel, which is closed at the second end of the system (i.e., at the downstream, distal end of the channel). A channel is any continuous path for fluid flow and may comprise a series of connected fluid-flow paths—for example, a channel may be relatively narrow and then may widen into a chamber, after which the channel narrows again; and a channel may comprise multiple chambers along its path. In the various embodiments described herein, it is immaterial how long the channel is, so long as the distal air is not vented. Such conditions ensure that the initial amount of air inside the cartridge, which is a known amount, does not change once the fluid sample is introduced or at any point in time thereafter during testing, as the air will be trapped by the fluid sample inside the cartridge, and will not leave the cartridge as the testing proceeds; the air is trapped on one end by the fluid sample and on the other end by the closed distal end of the channel. In some embodiments where the temperature is held constant, the pressure and the volume for the air trapped distal of the fluid sample at the beginning of the process (t=0) can be used to predict and control the position of the fluid sample at any time point later in the process (t=x; x>0) such that:

$$\begin{matrix}{P}_{0} {V}_{0} = {P}_{x} {V}_{x} \\ {V}_{x} = {P}_{0} {V}_{0} / {P}_{x}\end{matrix}$$

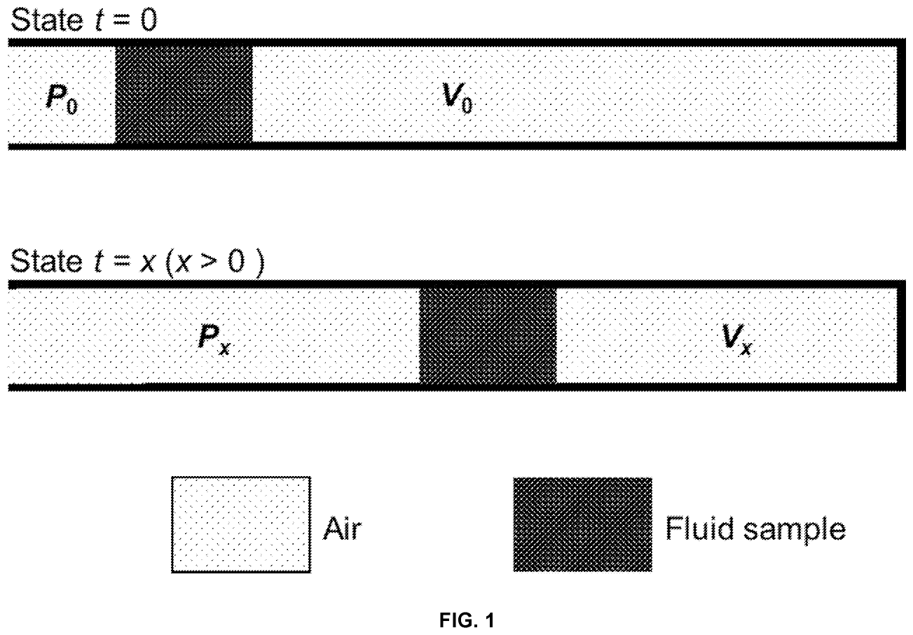

For example, by increasing the pressure Pₓ, the volume Vₓ is proportionately reduced. This principle, which is the principle underlying the operation of the air spring, is depicted in FIG. 1.

In some cases, a physical property of the fluid sample (e.g., the sample's viscosity) may change during testing, as the result of changes in temperature or reactions that take place in the sample, for example. A change in temperature may affect viscosity and in turn affect the velocity of the fluid sample's flow, especially in cases of fluids that exhibit non-Newtonian behavior, such as blood, etc. In addition, for non-Newtonian fluids, the fluid sample's viscosity may change as the sample moves along the path of a channel with changing dimensions and that thereby exposes the sample to different shear. Further, some testing conditions or reactions in the sample also may affect the pressure within the channel. For example, increasing temperature may lead to evaporation of some of the fluid sample within the channel and/or to fluid expansion (where the fluid occupies more volume). In some cases, the compressibility of the sample may affect movement of the sample through the pressurized channel. In order to create a robust, closed system that is suitable for multiple sample types and conditions, fluidic position sensors may be included within the cartridge, and the cartridge may contain an active feedback mechanism for controlling movement of the sample within the cartridge. In such cases, the fluidic position sensors can communicate with a compensation mechanism providing for an active feedback loop, where the system is pressurized or de-pressurized based on information regarding the fluid sample's position that is obtained from the position sensors; the pressurization and de-pressurization controls the sample's movement, such that the sample reaches the desired location within the channel at the desired time and maintains such desired location for a desired time period. The active feedback loop involving the position sensors, which can result in pressurization or de-pressurization of the channel, provides adequate control of the fluid sample's movement through and within the channel.

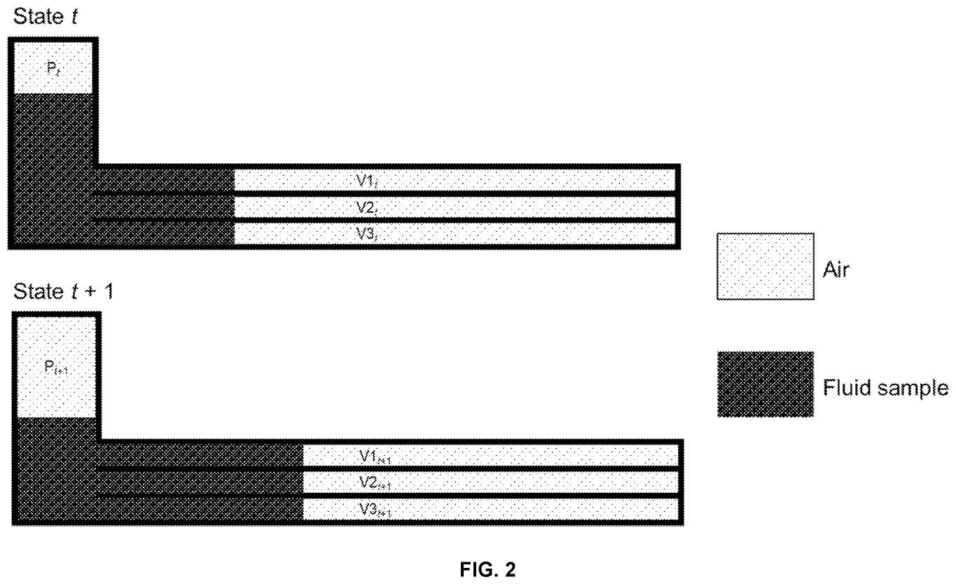

The process described above for a single channel can be applied to multiple channels to drive a fluid sample through multiple channels of a microfluidic cartridge using a single air source. For example, FIG. 2 shows the manner in which a microfluidic cartridge may receive a fluid sample through a common inlet connected to a first end of a plurality of channels. The sample in the common inlet is then split into multiple channels; certain channels contain an aliquot of the fluid sample, each aliquot having its own channel and each aliquot having a pre-determined volume, while another channel receives any excess fluid sample. The volumes of the aliquots may be the same or may differ. For example, in embodiments where the volumes of the aliquots differ, such different aliquot volumes can be achieved by using different metering well geometries, and/or by manipulating resistor regions in the channels to direct different amounts of the fluid sample to different metering wells.

Additionally, at any moment in time and for any given channel, the position of an aliquot of the fluid sample within a channel may be determined by the volume of the air distal to (i.e., downstream of) the aliquot. Using the volume of the distal air (which is the air downstream of the aliquot) to determine the location of the aliquot within a channel provides a way to determine aliquot location that is independent of channel geometry and other variables (e.g., viscosity of the fluid sample). Such method of determining aliquot location, and further of using an air spring design to adjust the location of the aliquot within the channel, provide several advantages over conventional microfluidic designs. For example, if the volume for the distal air trapped inside the cartridge is sufficiently large (such that the pressure in the system is low enough to allow for the fluid sample to move relatively easily, and to thereby provide for improved control over movement of the fluid sample portions), small variations between or among channels that would otherwise result in differing channel resistances and hence inaccurate testing results can be accommodated by using the air spring operation of the system. The air spring system as described herein can, for example, tolerate manufacturing defects that may result in non-identical channel geometries (where identical channel geometries are desired). Another advantage is that using an air spring system renders the cartridge suitable for different sample types, including fluid samples that differ in their physical properties (e.g., viscosities). Suitability across a broad range of different sample types is important in, for instance, blood diagnostics, since blood samples with varying hematocrits will have different viscosities and thus will move differently through a microfluidic cartridge.

Accordingly, unlike conventional systems, the air spring system described herein can accommodate a wide range of fluid properties (such as a wide range of viscosities, densities, compressibilities, etc.). For example, under the same conditions, a fluid of a higher viscosity may take longer than a fluid of lower viscosity to reach a given destination (e.g., the testing chamber) in the cartridge. The air spring system can accommodate such viscosity differences, and different rates of fluid movement, by adjusting pressurization within the channel(s) based on the location of the fluid sample within the channels and/or the volume of distal air, and thus a cartridge as described herein can be used for fluid samples that greatly differ in their physical properties (e.g., blood versus saliva). Similarly, with respect to a single fluid sample, the individual aliquots of the sample may acquire different physical properties while in the cartridge (e.g., due to each aliquot's exposure to different conditions within the cartridge, for example in channels having different reagents, as discussed herein); the air spring system can accommodate such different physical properties of the individual aliquots, by allowing for independent adjustments in each aliquot's position within the cartridge based on the volume of distal air and using the pressurization/de-pressurization feature discussed above.

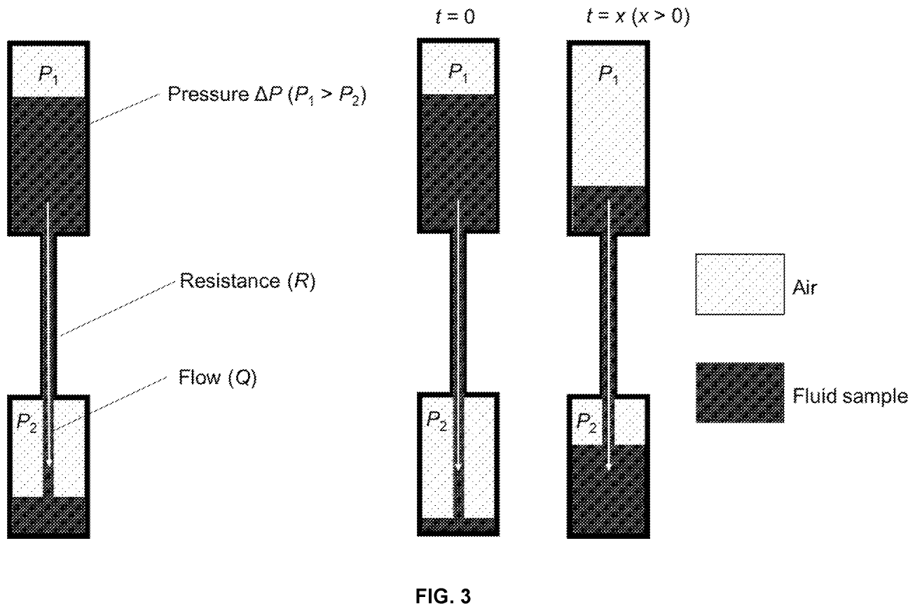

FIG. 3 illustrates time-dependent flow in a Hagan-Poiseuille system like the air spring microfluidic cartridge embodiments described herein. To ensure proper metering, and hence a known and determinable volume in each of the channels used for testing, the relationship between a pressure differential (ΔP), volumetric flow rate (Q), and resistance to fluid flow (μ) is given by:

$$Δ P = 8 μ LQ / π {r}^{4}$$

where r is the radius of a cross-section of a cylindrical microchannel. Some embodiments may use a rectangular microfluidic channel, in which case resistance (R) is related to u as well as the height (h), width (w), and length (L) of the microchannel, so that:

$$R = 12 μ L / {h}^{2} w$$

In order to perform consistent measurements and hence ensure accurate analyses across multiple channels of a microfluidic cartridge, a metering step first can be performed to ensure that each channel contains an aliquot of the fluid sample that has a specific, known volume. In some embodiments, this metering step further ensures that each aliquot of the fluid sample downstream of the metering is exposed to a known concentration of a reagent (or of multiple reagents). By applying pressure, the fluid sample will flow into and through each channel. In addition, the geometry of a channel can provide resistance to flow; such resistance may be localized to a specific region of the channel. Such resistance will create back pressure, and such back pressure may contribute to a pressure differential (the difference between P₁ and P₂ in FIG. 3). Such back pressure thus can serve as a way to slow the flow of the fluid in the channel. Accordingly, selecting certain channel dimensions (such as channel cross section and/or channel length) for the cartridge's channels is a way to further control the rate of movement of fluid through the channels. Flow will continue, and the fluid will continue to move downstream, provided there is a pressure differential (see, e.g., FIG. 3).

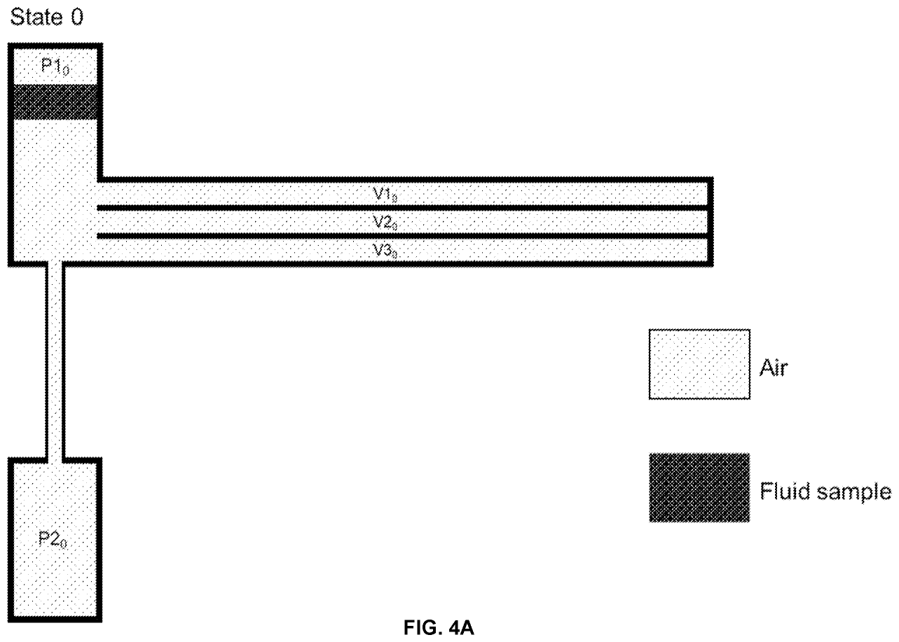

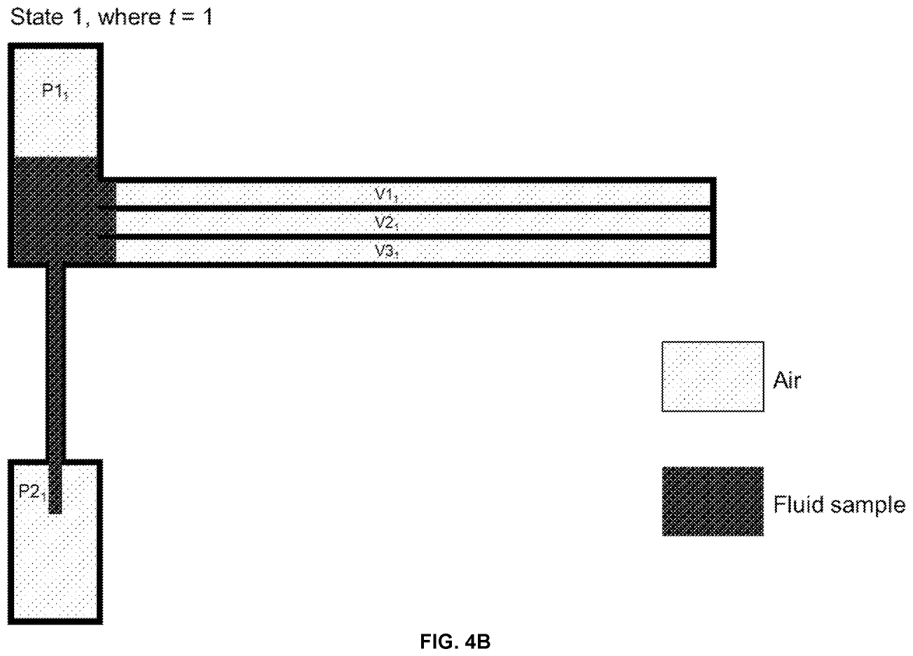

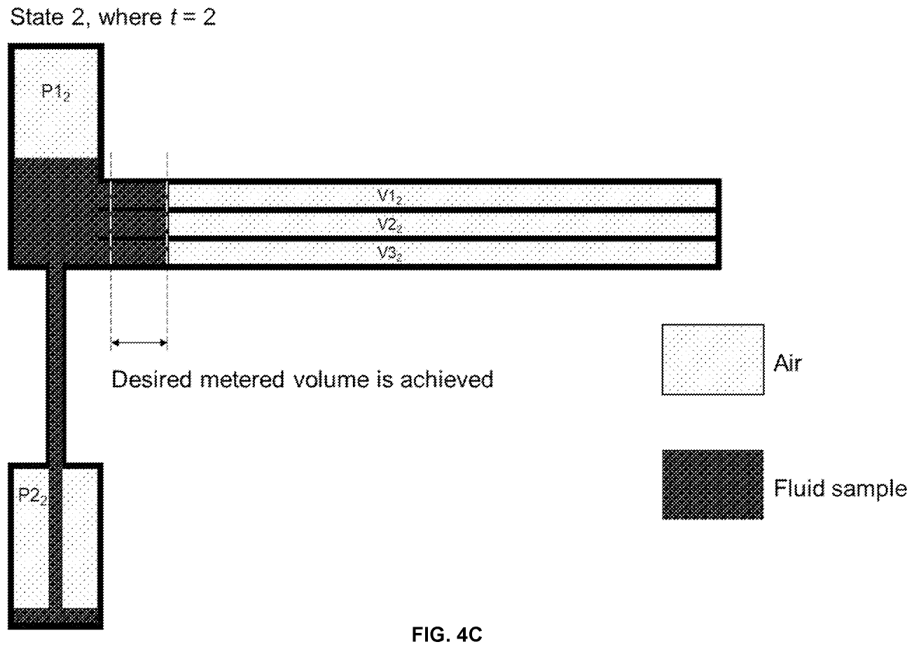

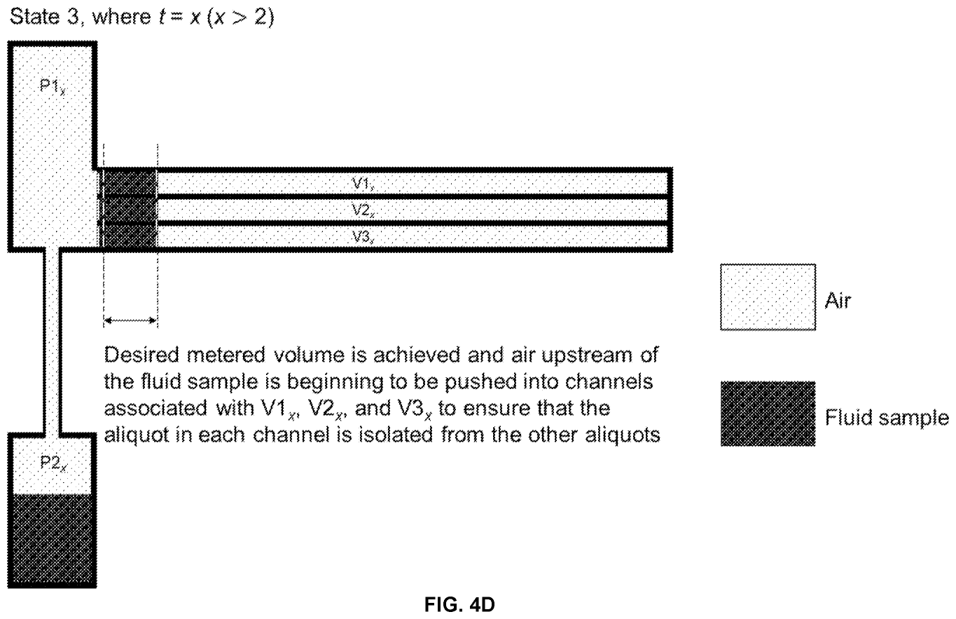

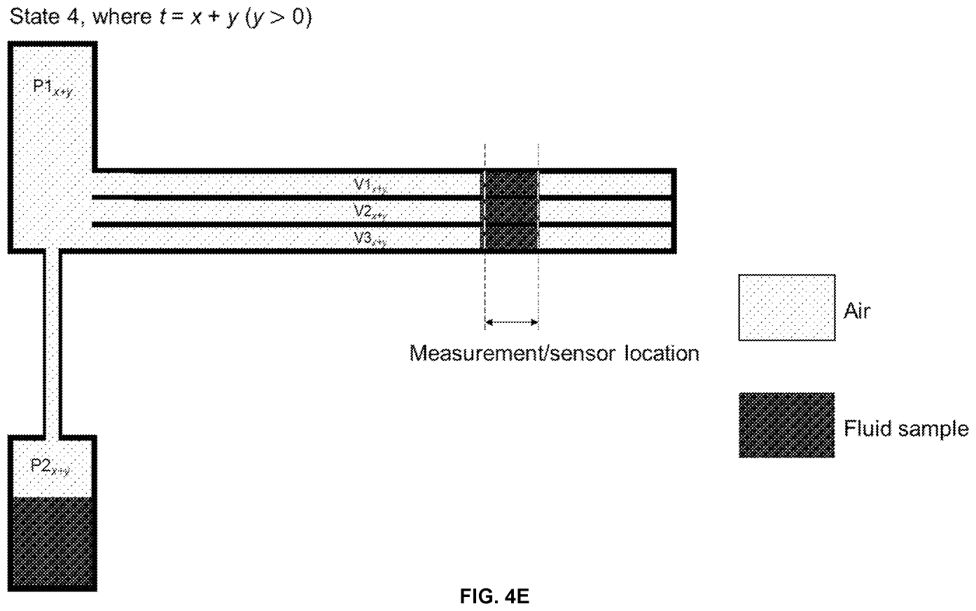

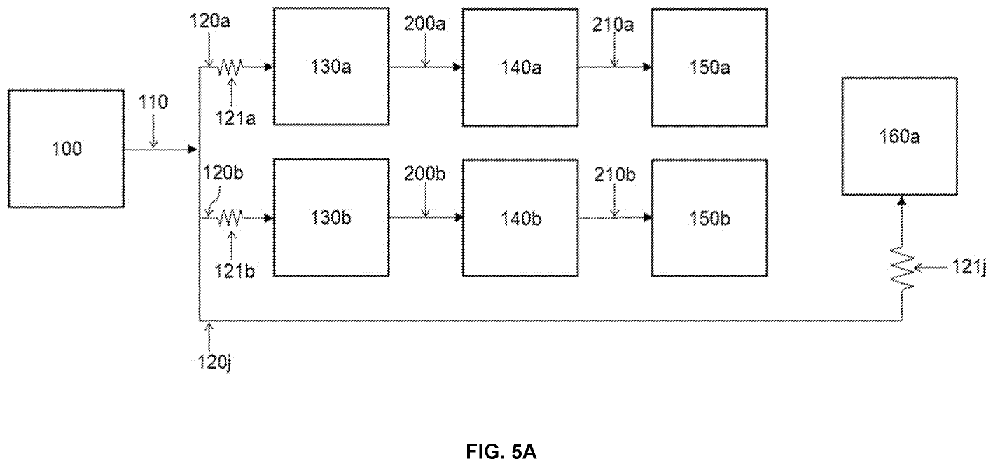

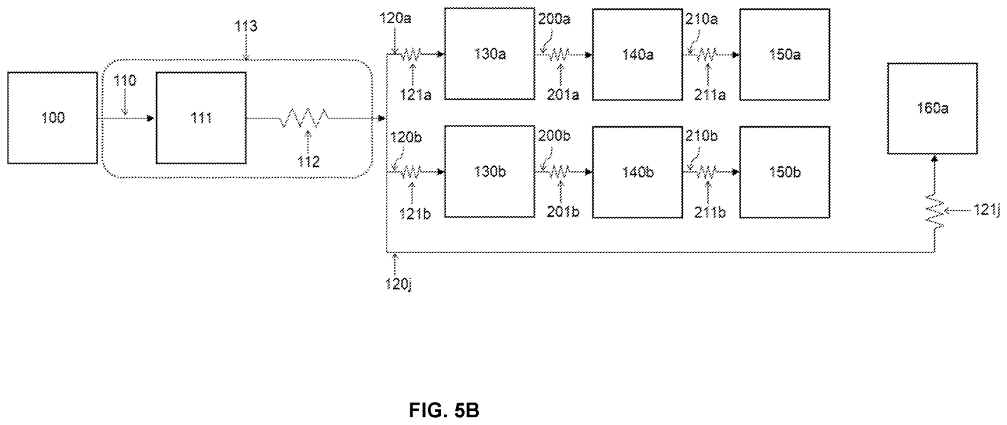

FIGS. 4A-4E are schematics depicting fluid placement and proximal and distal air, at progressing time-dependent states, in three channels of a single microfluidic cartridge. FIG. 4A depicts a cartridge at the time a fluid sample is inserted at a common inlet, and before the sample has split into the three channels and before any metering has occurred. In particular, FIG. 4A depicts a cartridge at state 0 (t=0), where all pressures inside the cartridge are at equilibrium and there is no pressure differential. Splitting and fluidic flow is subsequently accomplished in accordance with the ideal gas law and applying pressure to increase P₁. For example, FIG. 4B shows the same cartridge at state 1 (t=1), where the fluid sample is pushed to a known or predetermined position using the air spring. FIG. 4B further shows air trapped by the fluid sample in each of the three closed microfluidic channels; this air is distal (downstream) of the fluid sample portions in each channel and is associated with volumes V1₁, V2₁, and V3₁. The pressure associated with V1₁, V2₁, and V3₁ in each of the channels is equal to the pressure P1₁, whereas there is a pressure differential in the channel leading to P2₁ (i.e., P1₁≠P2₁). Over time (as pressure is applied at the inlet), pressure P1₁ becomes greater than pressure P2₁ and the fluid sample moves into the chamber associated with P2₁. Axiomatically, the total volume of the fluid sample inserted into the microfluidic cartridge (e.g., via inlet 100 in FIG. 5A) must be greater than the sum of all metered aliquots across each of the channels used for testing (e.g., the channels associated with V1₁, V2₁, and V3₁ in FIG. 4B; channels with chambers 130a and 130b in FIG. 5A). FIG. 4C illustrates a cartridge wherein the pressure is increased to a predetermined state (in state 2, designated at t=2) to fill each of the three channels with a desired fluid volume. Thereafter, the volume of fluid in each of the three channels increases, and as a result, the air in each of the three channels is compressed and V12, V22, and V32 each decrease; additional fluid is allowed to enter each channel until a desired fluid volume is achieved. At any moment in time when fluid is not moving, the pressure in each channel associated with distal volumes V1, V2, or V3 is the same as the pressure P1 and the pressure P2 (as the system drives toward pressure equalization). As P1 increases and becomes greater than P2 (while P1 is the same as the pressure associated with each of V1, V2, and V3), fluid sample will drain into the chamber associated with P2. After the desired metered fluid volume in each channel is achieved and the excess fluid sample resides in the chamber associated with P2, the air associated with P1 and that is upstream of the fluid sample aliquots (at the proximal side of the fluid) is pushed towards the channels associated with V1ₓ, V2ₓ, and V3ₓ, to ensure isolation of the aliquots in each of the channels associated with V1ₓ, V2ₓ, or V3ₓ; this point in the process (t=x; x>2) is illustrated in FIG. 4D. Air continues to push the aliquots through the channels until each aliquot reaches the measurement and/or sensing location (see FIG. 4E).

The devices, systems, and methods described herein can be applied to samples from any individual, including mammals (e.g., humans, such as human patients, as well as non-human mammals), reptiles, birds, and fish, among others, and can be useful for research and veterinary medicine. An individual can be, for example, mature (e.g., adult) or immature (e.g., child, infant, neonate, or pre-term infant). The devices, systems, and methods described herein can also be used for environmental samples (e.g., to test for the presence of an analyte in a water sample).

The devices, systems, and methods described herein can be used in diagnostics and/or prognostics. For example, the devices, systems, and methods described herein allow for multiplexed testing of a patient sample in a closed system. Such testing is particularly useful, for instance, in the context of infectious hemorrhagic diseases (e.g., Dengue virus, Marburg virus, Ebola virus, etc.), where a disease-specific cartridge can be used to both diagnose and predict the likelihood of hemorrhagic complications.

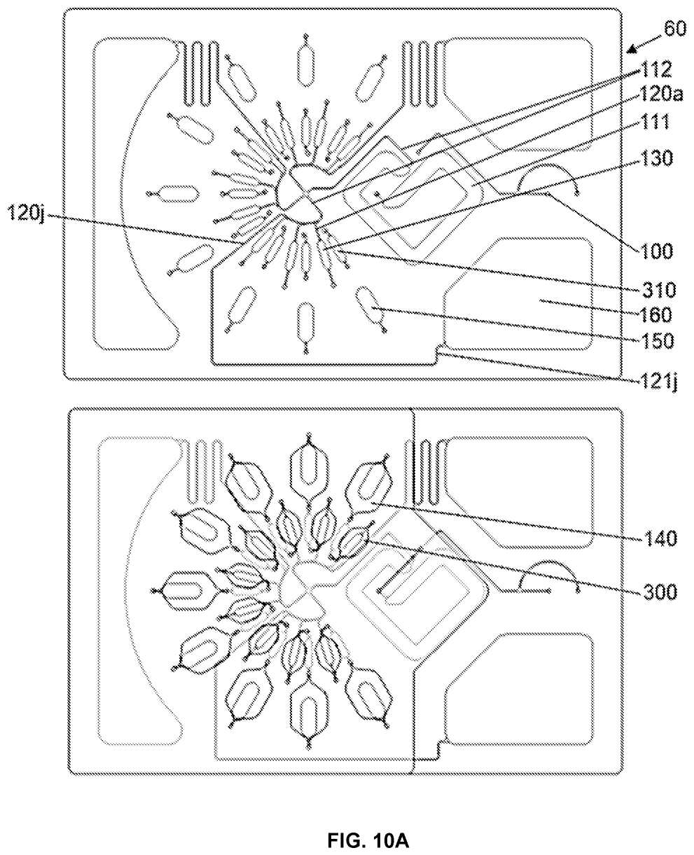

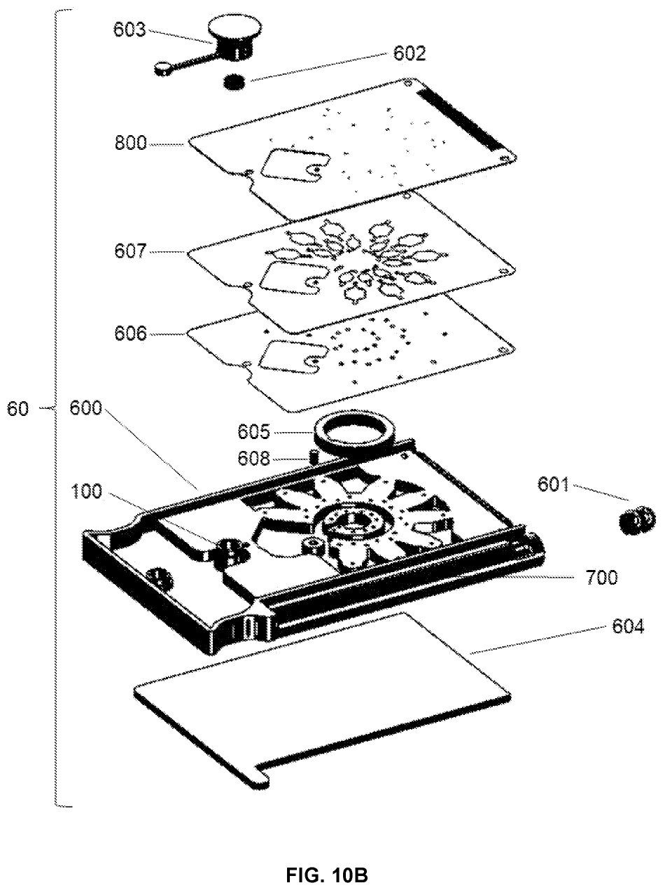

The devices, systems, and methods described herein can be used to guide therapy of a patient, including, without limitation, in the hospital setting. For example, physicians can use a multi-channel microfluidic cartridge that allows for multiplexed, independent testing of an aliquoted fluid sample without cross-talk between the channels (see, e.g., the cartridge embodiments of FIGS. 7, 10A-B) to perform, for example, the assays described in U.S. application Ser. No. 16/046,816 (published as US 2019/0111431) and U.S. application Ser. No. 17/195,615 in order to, for instance, ensure that patients requiring anticoagulation therapy are receiving an appropriate dose of anticoagulant so as to solicit the proper physiological response.

The devices, systems, and methods described herein likewise can be used in the management of diseases or disorders that result in pathological bleeding or clotting (e.g., COVID-19 caused by the SARS-COV-2 virus, hemophilia, etc.), including, without limitation, for the clinical and pre-clinical assessment of treatments (e.g., drugs).

The disclosed devices, systems, and methods also can be used for research and discovery. For example, the devices, systems, and methods described herein can be useful for basic drug discovery, for understanding disease or disorder pathophysiology, for pre-clinical assessment of new compounds and biologics, and for monitoring adverse events and off-target effects of experimental treatments.

### Examples

Various embodiments of a closed microfluidic cartridge comprising an air spring system are shown in FIGS. 5A, 5B, 6A, 6B, and 7.

The schematic in FIG. 5A depicts a basic closed cartridge system; the system comprises a plurality of channels (120), each channel of the plurality of channels (120a, 120b, and 120j) connected on a first end to a common inlet 100 via a common channel 110, and each channel of the plurality of channels having towards its second end a separate chamber or series of chambers (e.g., 130a, 130b, 160a). In some embodiments, a separate chamber in each of the channels may be a metering chamber 130 or a waste chamber 160.

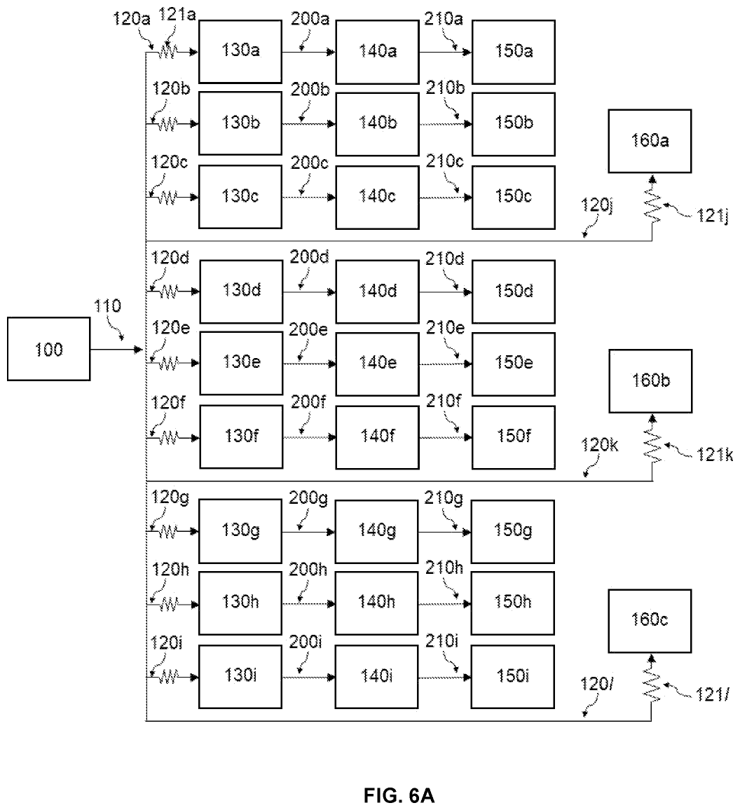

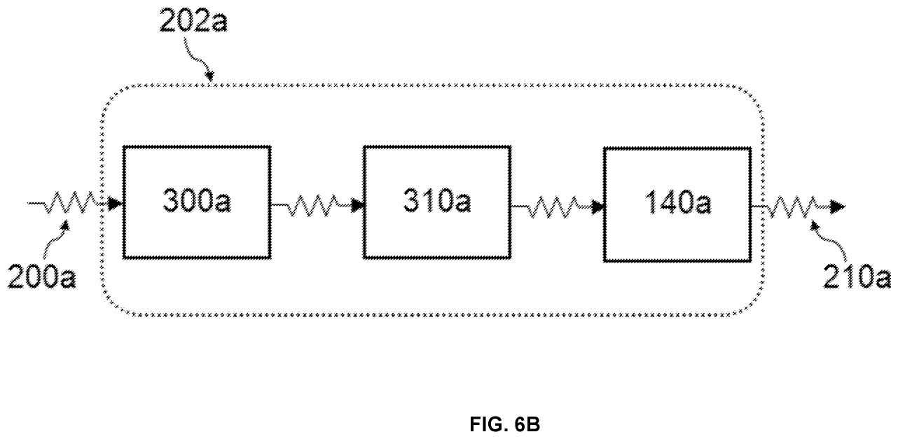

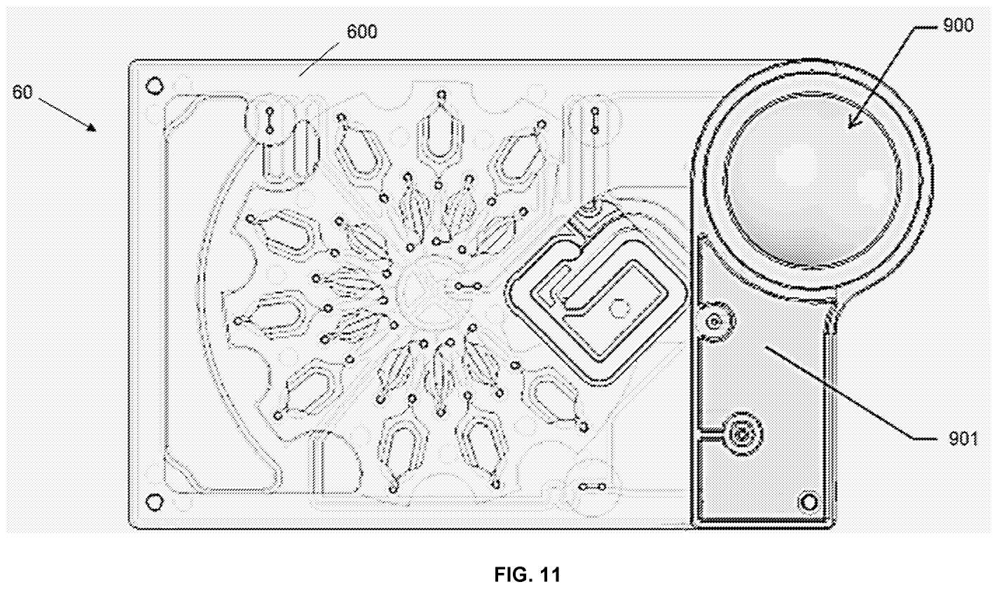

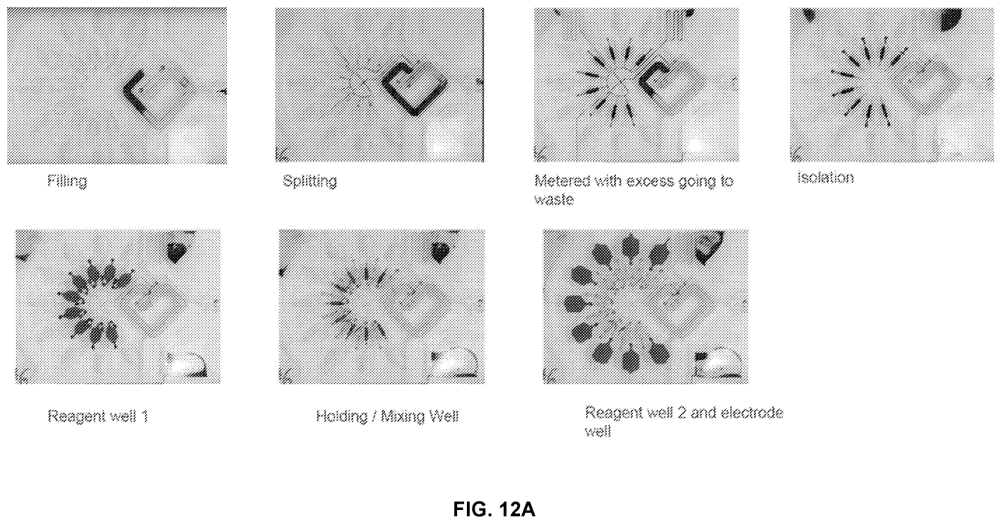

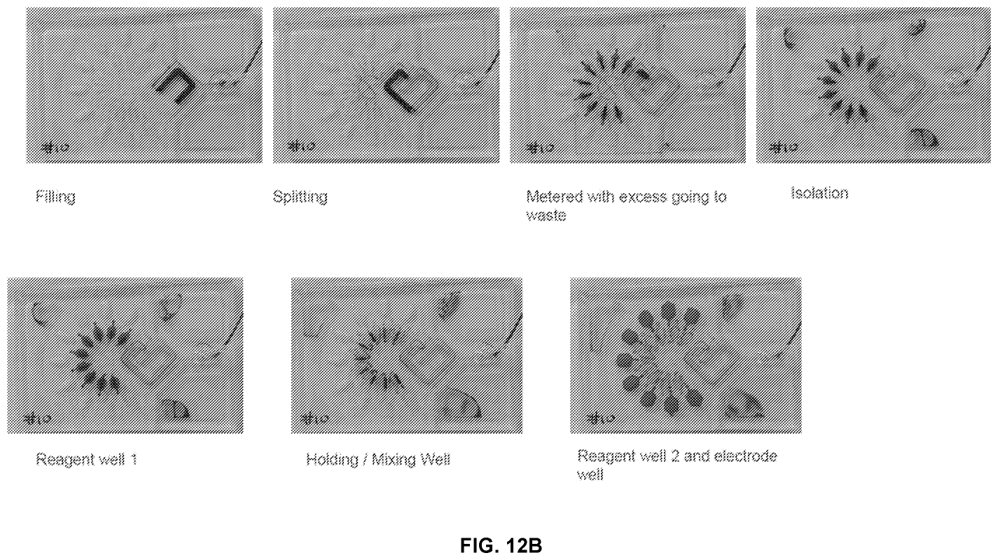

In a cartridge employing such a design, a fluid sample is introduced (e.g., inserted) via common inlet 100. Upon activation of an actuator (not shown in FIG. 5A) (see, e.g., feature 700 in FIG. 10B; feature 900 in FIG. 11), air upstream of the fluid sample is pushed, causing the sample to move along microfluidic channel 110 before splitting into channels 120a, 120b, and 120j. Each microfluidic channel 120a, 120b, and 120j contains at least one resistive element (121a, 121b, and 121j, respectively) that controls the rate of fluid flow in that channel, and thus into each of the chambers in that channel (e.g., chambers 130a and 130b and waste chamber 160a). In some aspects, such resistive features may include, for example, the geometric setup of the microfluidic channel (e.g., a change in the channel's aspect ratio or other change in channel geometry that reduces channel volume, etc.). In some embodiments, a resistive feature (e.g., 121j) may be, but need not be, physically distinct from the channel (e.g., the resistance imparted by 121j may be accomplished via, for example, the geometry of channel 120j, in which case, the resistive feature would not be distinct from the channel). In some embodiments, channel 110 may split into more than three microfluidic channels (see, e.g., FIGS. 6A, 7, 10A).

In various embodiments of the devices, systems, and methods described herein, portions of the fluid sample may be metered after the fluid sample is split across multiple channels, such that the metered portions have equal fluid volumes (metered volumes of the fluid sample are also called aliquots). In an example, the resistance of 121a is equal to the resistance of 121b, and 130a and 130b serve as metering chambers that accept the same volume of a fluid sample at the same rate. In some embodiments, where a channel (e.g., 110) splits into more than three microfluidic channels, it should be understood that there may be more than the two metering chambers illustrated in FIG. 5A (see, e.g., FIGS. 6A, 7, 10A). For example, the microfluidic cartridges of FIGS. 6A, 7, 10A, and 11 each show nine metering chambers (130a-130i). Generally, each channel that is used for testing will include a region that is a metering chamber.

In the cartridge embodiment shown in FIG. 5A, downstream of the metering chamber 130 is a testing chamber 140 (which is also called a sensing chamber or measurement chamber). In such an embodiment, each testing chamber 140a and 140b may contain sensors (e.g., electrode sensors) and/or may facilitate some other method of measurement (e.g., optical detection). Sensors may be contained within a channel or may reside outside of a channel. As described herein, sensors may include any component that allows for measuring or that otherwise measures characteristics of the fluid sample, including, for example, by measuring electrical impedance, measuring electrical capacitance, measuring electrical resistance, assessing flow rate (e.g., by assessing the flow rate of beads that are present in the fluid sample), measuring flow velocity, measuring pressure, measuring viscoelasticity, fluorescence detection (e.g., using fluorescent fibrinogen), measuring turbidity, infrared light detection, infrared spectroscopy, detection using acoustic sensors, detection using photonic sensors, flow cytometry, and methods of visual detection (e.g., for visual clot detection).

In some embodiments, one or more testing chambers 140 may also contain a reagent (including, e.g., multiple reagents). Reagents may include coagulation factors, calcium, fluorogenic or chromogenic substrates, coagulation activators (e.g., glass, celite, phospholipids, kaolin, etc.), electrochemical reagents (e.g., for thrombin generation, etc.), and platelet activators (e.g., adenosine phosphate, collagen, ristocetin, epinephrine, etc.).

As shown in FIG. 6B, in some embodiments, there is a chamber (or a series of chambers) between metering chamber 130 (e.g., 130a) and testing chamber 140 (e.g., 140a) in each of the channels used for testing. In such embodiments, at least one of these additional chambers may contain one or more reagents.

Each testing chamber (or well, etc.) 140 (e.g., 140a) connects to a distal air spring 150 (e.g., 150a) via a chamber (or a series of chambers) 210 (e.g., 210a). Each air spring is a closed well of a predetermined volume such that it and the waste chamber(s) (160) control fluidic movement. In designing cartridge embodiments as described herein, the volume of the air springs can be increased; such increase in volume will decrease the pressure of the system (including the pressure of the system when the aliquots are at their final destination in the testing chambers). Alternatively, the volume of the air springs can be decreased; such decrease in volume will increase the pressure of the system (including the pressure of the system when the aliquots are at their final destination in the testing chambers). Similarly, increasing or decreasing the volumes of each waster chamber (160) will affect how much pressure is within the cartridge and the air spring system.

In various aspects, a cartridge may comprise more than two metering chambers 130a and 130b and/or more than a single waste chamber 160a. Exemplary embodiments of such cartridges are illustrated, by way of example, in FIGS. 6A and 7. It should be understood by those skilled in the art that embodiments of a cartridge comprising a closed air spring system may include features that are not depicted in FIG. 5A (see, e.g., FIGS. 6A, 7), and/or that are not shown in the specific embodiments described herein.

In some embodiments, it may be desirable to split the fluid sample in multiple, successive steps. For example, successive or modular splitting may improve metering time and/or fluidic fill across multiple channels. Such improvements may be important in embodiments wherein the fluid sample is split into a large number of portions. In some embodiments, the fluid sample is split into three separate portions, each portion moving into its own channel, and each of the three separate portions is then subsequently split across three or more metering chambers and one or more waste chambers.

In certain aspects, the embodiment shown in FIG. 5A may include additional features, such as the features of area 113 shown in FIG. 5B. In such embodiments, the inlet 100 connects to a reservoir 111, and resistor 112 controls and/or prevents backflow of the fluid sample. Reservoir 111 may serve, for example, as an additional metering region prior to splitting the fluid sample into multiple metering chambers and one or more waste chambers. In some embodiments, resistive feature 112 may comprise, for example, a tesla valve, a meandering microfluidic path, and/or a porous membrane. In some embodiments, it may be desirable to combine multiple resistive elements (e.g., multiple resistor regions in parallel or in series). In addition, in some embodiments, the paths connecting each of the chambers in a channel (e.g., 130 to 140 via 200; 140 to 150 via 210) may also include resistive features 201 and/or 211 (see, e.g., FIG. 5B). In some aspects, resistance to fluid flow may be achieved via the geometry (including shape and/or dimensions of that shape) of one or more of the paths between chambers (e.g., 200, 210 (including only at certain regions thereof)), such that there is no distinct corresponding resistor separate from the channel itself.

In certain embodiments of the cartridges described herein, the fluid sample may be split into at least twelve channels: at least nine channels (120a-i), each channel containing an aliquot of the fluid sample, and three waste channels (120j-l); see, for example, the embodiment depicted in FIG. 6A. The fluid sample may split into the at least twelve channels at a single time, or the fluid sample may first split into less than twelve portions, followed by subsequent splittings (e.g., the fluid sample may split twice, three times, four times, etc.). For example, in the embodiment of FIG. 6A, the common inlet 100 connects to microfluidic channels 120a-l directly via common channel 110 (compare, e.g., FIG. 5A). In another embodiment, such as the embodiment of FIG. 10A, the fluid sample splits into three channels, each such channel splitting further into three channels (see the channel labeled 120a) ahead (upstream) of metering chamber 130.

In various embodiments, the fluid sample may flow to a first fluid reservoir 111 and/or resistor 112 before splitting (see, e.g., FIGS. 5B, 7). As is illustrated in FIG. 6B, in some embodiments, one or more of the channels in a cartridge may further include additional paths (e.g., additional chambers, or a series of additional chambers) and/or resistive features between the metering chamber (130) and the testing chamber (140). In some aspects, each of the microfluidic channels (at one more locations along each channel's path, e.g., 120, 200, 210) may, but need not necessarily, include resistive elements. FIG. 7 provides a schematic for an embodiment comprising resistive elements at various locations along channel paths.

In certain embodiments where the cartridge comprises additional chambers and/or resistive features in between a metering chamber (130) and a testing chamber (140), each of the additional chambers and/or resistive features between the metering chamber (130) and the testing chamber (140) in each of the channels of the cartridge (e.g., the cartridge depicted in FIG. 7) may serve a particular purpose or provide a particular function. For example, as described above, one or more chambers in between a metering chamber (130) and a testing chamber (140) may contain one or more reagents.

In certain embodiments (e.g., the embodiments shown in FIGS. 6B, 7), the closed microfluidic cartridge and the various chambers and features included in the channels therein are designed to allow:
a. splitting of a fluid sample into multiple channels for analysis (e.g., 120a-i) and at least one waste channel (e.g., 120j);
b. isolating each of the portions of the fluid sample in each of the channels for analysis (e.g., 120a-i), and thereby separating such portions from the portions of the fluid sample in each of the waste channels (e.g., 120j-1) and thus in each of the waste chambers (160a-c), and further
c. separating each of the portions of the fluid sample in each of the channels for analysis (e.g., 120a) from each of the other portions of the fluid sample in each of the other channels for analysis (e.g., 120b-i);
d. metering the portion of the fluid sample in each of the channels for analysis (such as in chambers 130a-i), which can ensure that each portion in the chambers downstream of metering (e.g., reagent chambers, testing chambers (or wells, etc.)) is of a known volume (such volumes across channels may or may not be equal to each other);
e. introducing each of the portions of a known volume (called aliquots) to one or more reagents (such as in a chamber as depicted by 300);
f. mixing each aliquot of the fluid sample with the one or more reagents (such as in chamber 310); and
g. measuring or otherwise analyzing one or more physical and/or chemical characteristics of each aliquot of the fluid sample (such as in chamber 140), which permits evaluating each aliquot's reaction in response to the one or more reagents.
   FIGS. 6B and 7 show such a cartridge, which may be used, for example, to detect and/or evaluate blood clotting.

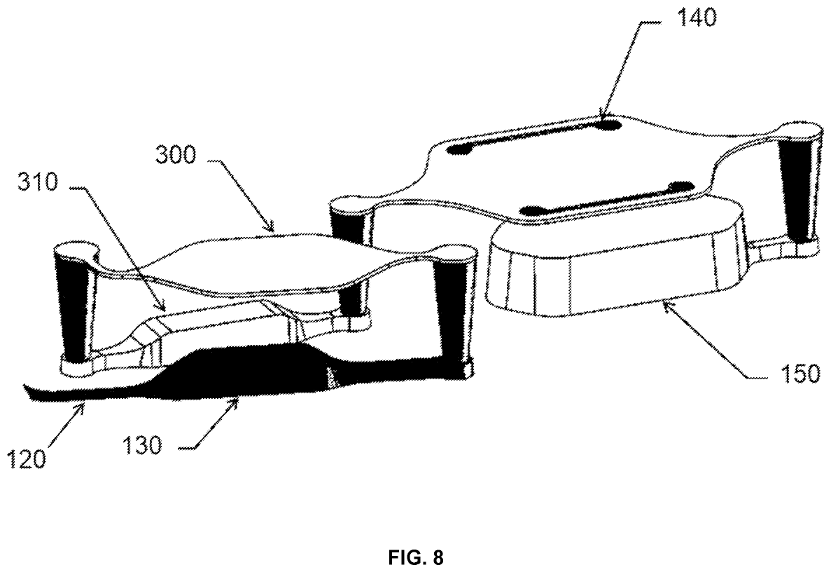

In some embodiments, a cartridge may comprise multiple, stacked layers, and the cartridge's channels and/or chambers may be made of different materials and/or reside along different planes. FIG. 8 shows one such example, where the chambers along the path of one of the microfluidic channels in FIG. 7 (e.g., the channel comprising 120a, 130a, 300a, 310a, 140a, and 150a) are shaped and positioned in three dimensions, such shapes and positions achieving various functions (e.g., items a through g described above). In a preferred embodiment, the reagent chamber and mixing chamber are sized and positioned relative to one another, as well as to the channel path connecting them, such that the reagent chamber 300 may accommodate up to 1.10 μL of an aliquot of the fluid sample, the mixing chamber 310 may accommodate up to 2.55 μL of an aliquot of the fluid sample, and channel path 400 connecting them may accommodate up to 0.85 μL of an aliquot of the fluid sample. In some embodiments, the volume of the mixing chamber (and the volume of the fluid sample aliquot in the mixing chamber) may be up to five times, or as little as 1.5 times, larger than the volume of the reagent chamber (and the volume of the fluid sample aliquot in the reagent chamber). In various aspects, the ratio of the volume of the fluid sample aliquot in the reagent chamber to the volume of the fluid sample aliquot in the mixing chamber may depend on, e.g., the hydroscopic behavior of the one or more reagents, the properties of the fluid sample, and/or the properties of the interior surface of the reagent chamber (e.g., hydrophobic, hydrophilic, patterned, etc.).

In certain embodiments, the configuration depicted in FIG. 8 ensures that the one or more reagents are fully mixed with the aliquot of the fluid sample through a process of power-washing, which takes place in the reagent chamber and which is then followed by mixing in the larger volume mixing chamber 310, which is connected to the testing chamber 140.

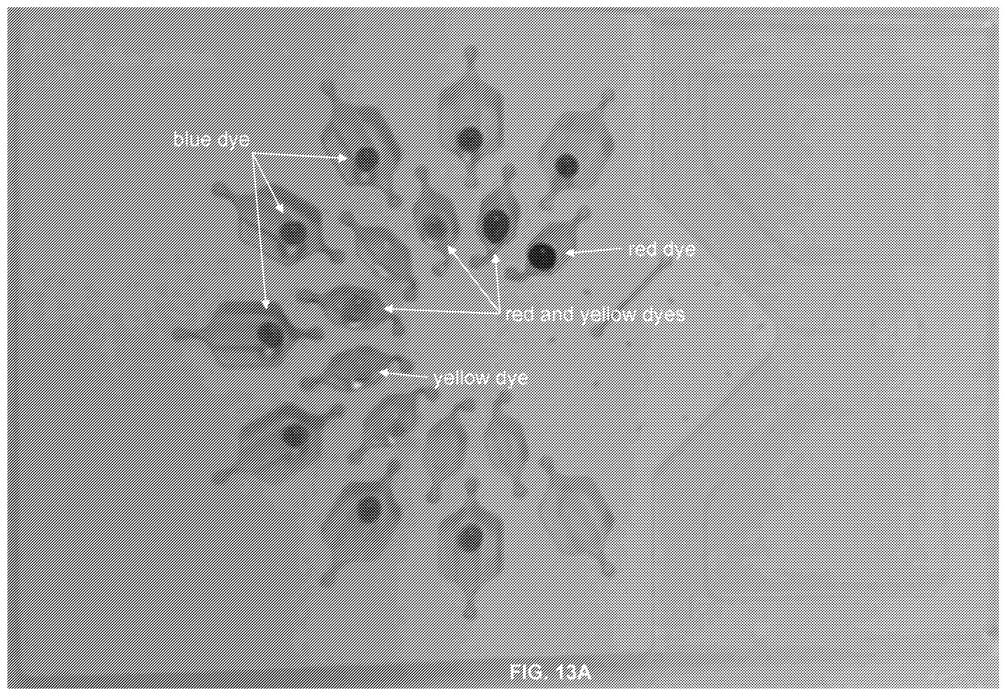

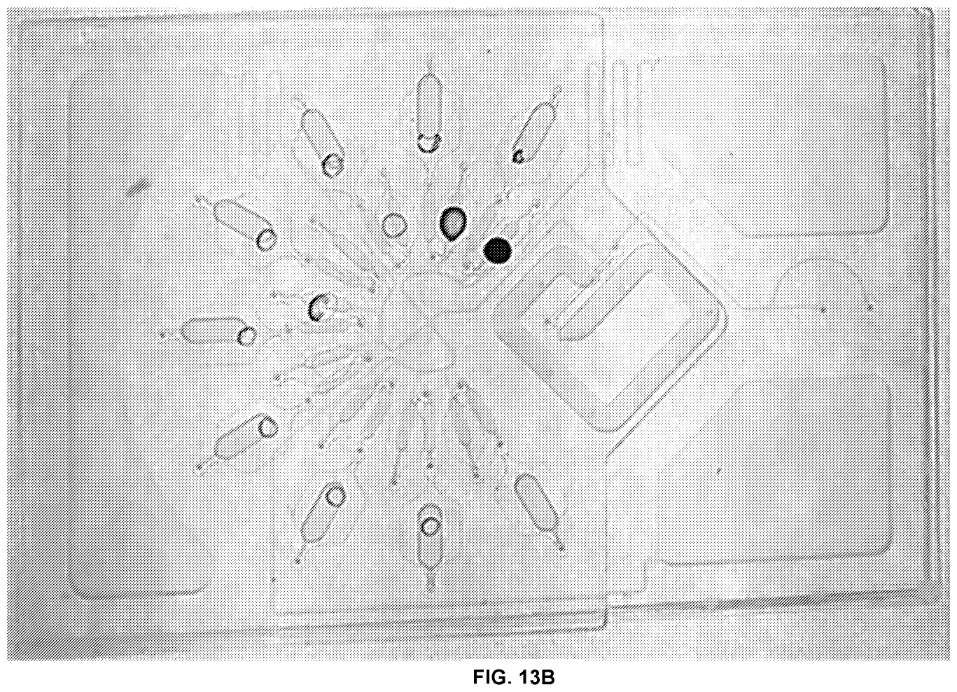

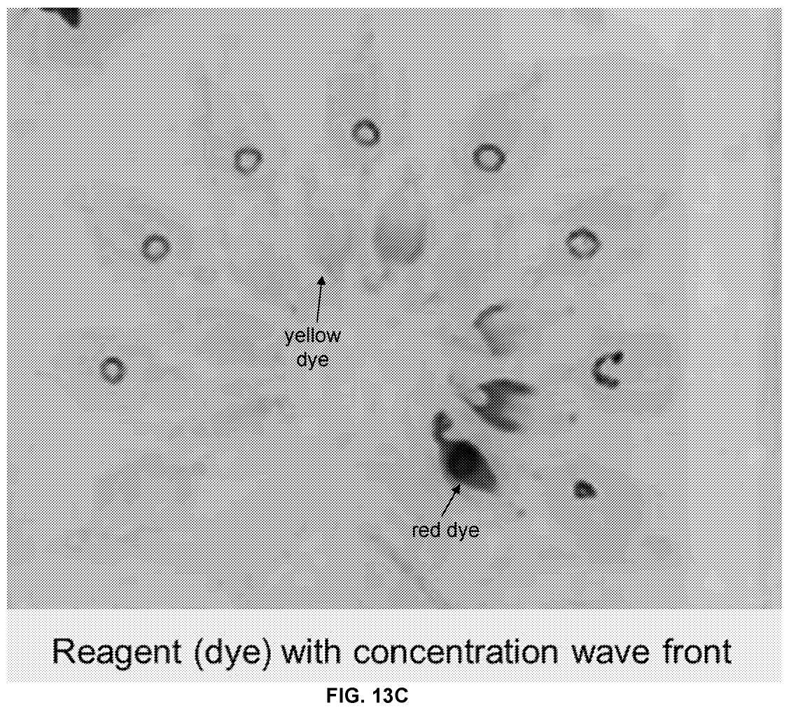

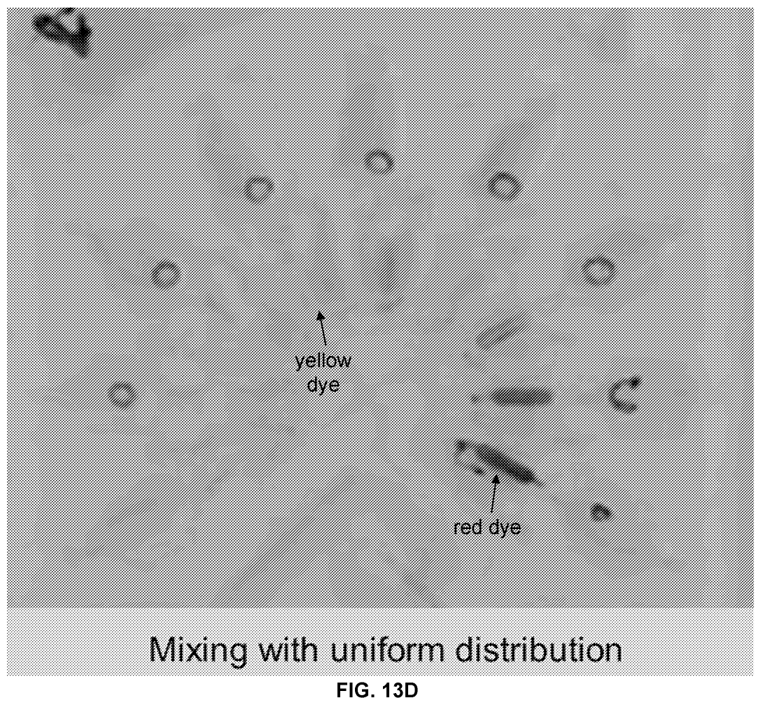

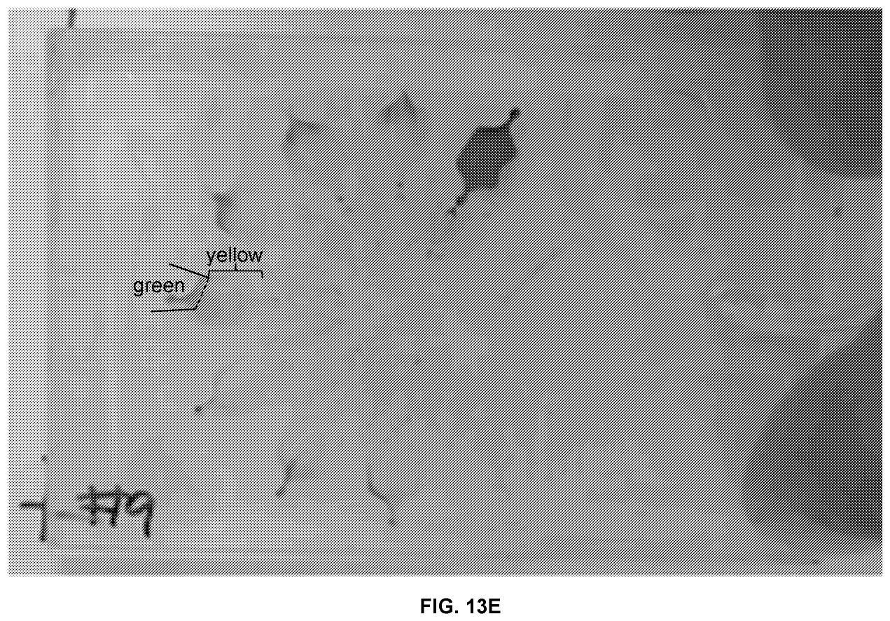

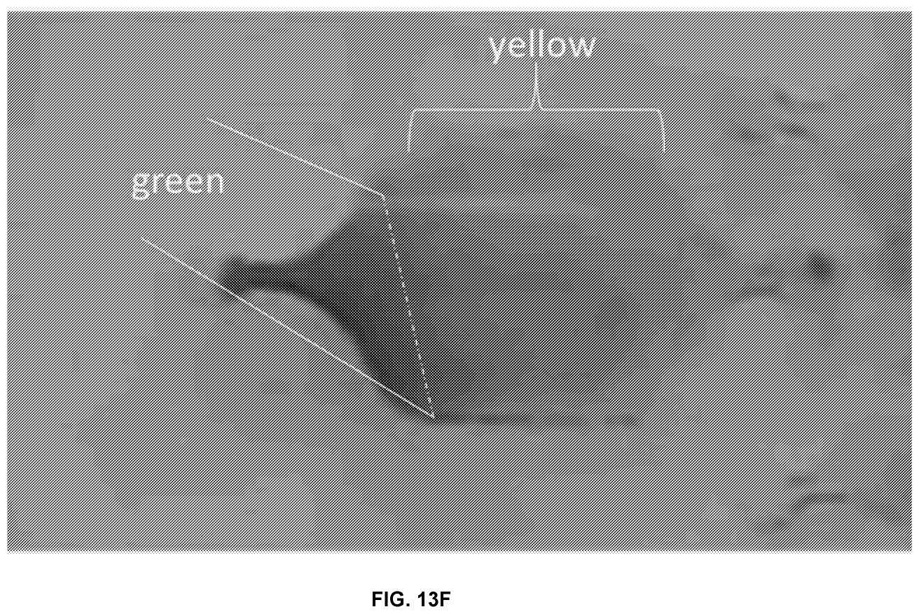

In some embodiments, this process of power-washing followed by mixing may be particularly desirable, for example, where one or more reagents have been dry-spotted on or within the microfluidic cartridge. The efficacy of power-washing is illustrated in FIGS. 13C-13F.

FIG. 13A shows the spotting of droplets with different color dyes and FIG. 13B shows the same droplets once dried (“dry-spotted”) and covered with a polycarbonate sealing layer that is placed on top to completely form the channels. The differently-colored dyes are dry-spotted in the reagent chambers, and each reagent chamber contains a red and/or yellow dye (at different concentrations). The testing chambers contain spots of blue dye. There are no dye spots in the mixing chambers, which cannot be seen in FIG. 13A. FIGS. 13C and 13D show images as water is flowing through the microfluidic channels in the cartridge. FIG. 13C shows a color gradient in the reagent chambers, as water flows through and brings the dried colored spot (the dried dye of red and/or yellow) into solution; the dried dye is used to represent an example reagent such as a coagulation agonist. The color dye fully mixes with the fluid sample (which, in this demonstration, is water) when each fluid sample aliquot is in the microchannel's mixing chamber, as shown in FIG. 13D (which shows dye dissolved uniformly throughout the water within the mixing chambers). This demonstration using dye also shows that there is no color mixing or bleed-over between the nine channels through which fluid is flowing in parallel; in other words, there is no cross-talk between the channels, and thus the multiple aliquots can be tested simultaneously. FIGS. 13E and 13F show images of the cartridge when each water aliquot with the red and/or yellow dye mixed therein has been pushed farther downstream into each microchannel's testing chamber containing the blue dye.

FIGS. 13A-13F demonstrate two important concepts: 1) the dye color(s) used to represent the reagents (the red and/or yellow dyes) in each microchannel is fully mixed with the fluid sample aliquot by the time the aliquot, with the reagent(s), reaches the testing chamber (see FIG. 13D), and 2) the reagent that was dry-spotted directly into the testing chamber (represented in this illustration with the blue spot) does not fully mix with the fluid sample aliquot in each microchannel, as seen by the blue color gradient in each testing chamber (see FIGS. 13E, 13F). FIG. 13E shows the testing chambers, with each testing chamber having a blue dye color gradient. For the microchannel with the reagent chamber that contained red dye to represent the reagent, if the red dye had fully mixed with the blue spot in the testing chamber, the resulting color in the entire testing chamber should have been purple, but it was not. Similarly, for the microchannel with the reagent chamber that contained yellow dye to represent the reagent, if the blue spot in the testing chamber had fully mixed with the fluid sample, the resulting color in the entire testing chamber should have been green (a combination of yellow from the reagent in the reagent chamber and blue from the testing chamber reagent), but it was not (see also FIG. 13F). Instead, only a relatively small portion of the testing chamber was green, and the majority of the testing chamber retained the color of dye from the reagent chamber. This illustration confirms that power-washing in a reagent chamber followed by mixing in a mixing chamber provides good reagent mixing with the aliquot by the time the aliquot reaches the testing chamber.

In some embodiments, a cartridge (such as 60 in FIG. 10B) may comprise an array of components. For example, a cartridge may comprise a cartridge body 600 that may include a plunger 601, filter plug 608, support ring 605, and a septum 602. The cartridge may further comprise a first pressure sensitive adhesive (PSA) layer 606, a second PSA layer 607, and printed circuit board 800. The PSA layers 606 and 607 and printed circuit board 800 additionally may be aligned with each other and with the cartridge body 600, and adhered or bonded (e.g., heat sealed, heat welded, laser welded, etc.) to each other and the cartridge body 600. The first PSA layer 606 and second PSA layer 607 may have either single-sided or double-sided adhesive. In some embodiments, a back seal 604 may also be bonded to the bottom of the cartridge body 600 to fashion the microfluidic channels.

In some embodiments, a fluid sample is moved through the microfluidic cartridge by pushing air in the cartridge after insertion of the fluid sample. Air can be pushed via an integrated plunger 601 (see, e.g., FIG. 10B). For example, an actuator may push on a plunger 601, such that the plunger 601 travels the length (or a portion of the length) of an integrated syringe 700 that moves the fluid sample to the desired locations within the cartridge at specific moments in time. In some embodiments, a cartridge may alternatively be outfitted with part 901, which includes a bellows 900 that operates to move the fluid sample through the cartridge (see, e.g., FIG. 11).

Various materials (e.g., acrylic, polycarbonate, glass, silicone, etc.) and fabrication methods can be used to make the cartridge body 600. For example, one or a combination of existing fabrication methods (e.g., injection molding, machining, laser etching, layering (which may include any combination of the foregoing, with or without photoablation and/or sacrificial layers)) may be used. In some embodiments, the tests and testing conditions (e.g., reagent(s) to be used, temperature, etc.) that will be used will inform the selection of material(s) to use in manufacturing the cartridge body.

In various aspects, a cartridge may comprise one or more layers of PSA. In some embodiments, a PSA layer may allow for creating fluid connections between layers (and different planes) of the cartridge (in such instances, a PSA layer with a particular pattern of openings can be used). PSA layers can be mass produced and cut with commercial laser cutters. Small production runs can also be achieved with off-the-shelf laser cutters, depending on the desired resolution of the openings. In addition, depending on the pattern and geometry (or geometries) of the openings, PSA layers may be die cut to create the desired pattern and/or geometry.

In embodiments where the cartridge comprises a printed circuit board (PCB), such as PCB 800 shown in the embodiment of FIG. 10B, such PCB can be rigid or flexible, or may comprise layers with one or more of such layers being flexible and other layer(s) being rigid. In an exemplary embodiment, a flexible PCB made of Kapton® is attached to (e.g., adhered to, laser welded to, etc.) a rigidized layer (such as a rigid PCB). In some embodiments, a flexible PCB allows for fluorescent measurements. PCB 800 also may contain heating elements within the electronics. Including heating elements may be desirable for coagulation testing or polymerase chain reaction (PCR) testing, for example. One or more temperature sensors and/or heating elements can be integrated into the PCB, to achieve and/or maintain a desirable temperature in one or more particular regions of the cartridge, such as over the regions of the cartridge containing the fluid sample. In some embodiments, each testing chamber may have its own temperature sensor and/or heating element. In further embodiments, localizing individual temperature sensors and/or heating elements to individual regions of a cartridge allows for exposing each aliquot to a different temperature and/or applying different temperatures to different portions of each channel. Temperature measurements also may be used by a testing unit for a feedback temperature control system.

Cartridges having closed systems, wherein each of the aliquots of the fluid sample is isolated, as described herein, may be used to perform multiple tests (e.g., to assess coagulation in each of the aliquots) simultaneously. In embodiments involving coagulation tests, such tests may involve assessing activated clotting time, prothrombin time, activated partial thromboplastin time, dilute thrombin time, or thrombin time. Such tests may involve coagulation factor activity assays, chromogenic assays (e.g., to assess anti-Factor Xa activity), fluorescence-based assays (e.g., fluorescent fibrinogen-based assays), electrochemical-based assays (e.g., thrombin generation), and any of the functional assays disclosed in U.S. application Ser. No. 16/046,816 (published as US 2019/0111431) and U.S. application Ser. No. 17/195,615, the contents of each of which are incorporated by reference in their entirety herein.

In some embodiments, electrochemistries of the fluid sample aliquots can be evaluated, such as, for example, for the measurement of one or more electrolytes (e.g., glucose or lactate, etc.) and/or biomarkers (e.g., genetic or protein biomarkers). Such analysis can be achieved with the addition of special reagents, use of electrode coatings, and/or electrode functionalization.

In some embodiments, enzyme-linked immunosorbent assays (ELISAs) can be performed in the microchannels, such as for the detection and quantification of one or more biomarkers and/or antibodies. ELISAs can be performed using electrodes that may or may not be functionalized (e.g., directly with antibodies, with nanogold or microgold particles, and/or with carbon nanotubes), or may be performed using agglutination, fluorescence, magnetic beads, chromogenic methods, sensors within or outside of the testing chambers, or any combination thereof.

Isolating each of the fluid sample aliquots, such that each aliquot moves through a single microchannel, makes it possible, in some embodiments, to use a single cartridge to perform multiple platelet activation and inhibition tests, including aggregometry, adenosine triphosphate (ATP) generation (e.g., via luciferase measurements), platelet adhesion (e.g., using imaging, or mechanical and/or electrical sensors), and/or platelet counts (e.g., via imaging and/or with other sensors). In further embodiments, both coagulation and platelet tests can be performed using a single cartridge.

In some embodiments, PCR can be performed—for example, traditional PCR with thermocycling or isothermal reactions, or newer PCR methods, such as those using clustered regularly interspaced short palindromic repeats (CRISPR).

Various types of sensors may be incorporated into the testing chambers in order to perform any of the tests described above or that otherwise may be desired. Such sensors include, for example, gas sensors that can be used to detect pathogens, toxins, volatile organic compounds (VOCs), or other hazardous chemical or biological agent. In some embodiments, such sensors may be used to evaluate the chemical composition of the fluid sample within the testing chamber. Other embodiments may involve electrode sensors, as discussed above. In addition, in some embodiments the cartridge may comprise sensors in addition to the sensors used for evaluating the sample aliquot in the testing chamber. Such additional sensors may be placed at points along the fluid path that are not within the testing chamber (e.g., an additional sensor may be located upstream of the testing chamber). Further, additional sensors may be positioned inside the channel or outside of the channel. For example, in the microfluidic device of FIG. 10A, an electrode pair that can be used for evaluating the amount of hematocrit in a blood sample may be included, as shown in FIG. 9.

In some embodiments, a material used to manufacture a cartridge may be transparent in order to allow optical measurements and imaging of the fluid sample aliquots within the channels. For example, for a multi-layer cartridge, one or more layers may be transparent or may otherwise allow for optical measurements or imaging. Such optical measurements or imaging can take place at one or more locations of the channels, including at locations that are not testing chambers. In addition, such cartridges may or may not comprise electrode sensors for measuring an electrical property (e.g., electrical impedance) of the sample aliquots.

In some embodiments, testing may require exposing one or more of the sample aliquots to multiple reagents at various times (e.g., by first exposing an aliquot to one reagent, followed by exposure of the aliquot to another reagent) and may also require heating and/or cooling of the fluid sample aliquot(s). For example, coagulation testing may require heating the cartridge containing the fluid sample to 37° C.; in further embodiments, testing may require heating the cartridge and/or the portions of the fluid sample to the body temperature of the patient, to approximate physiological conditions.

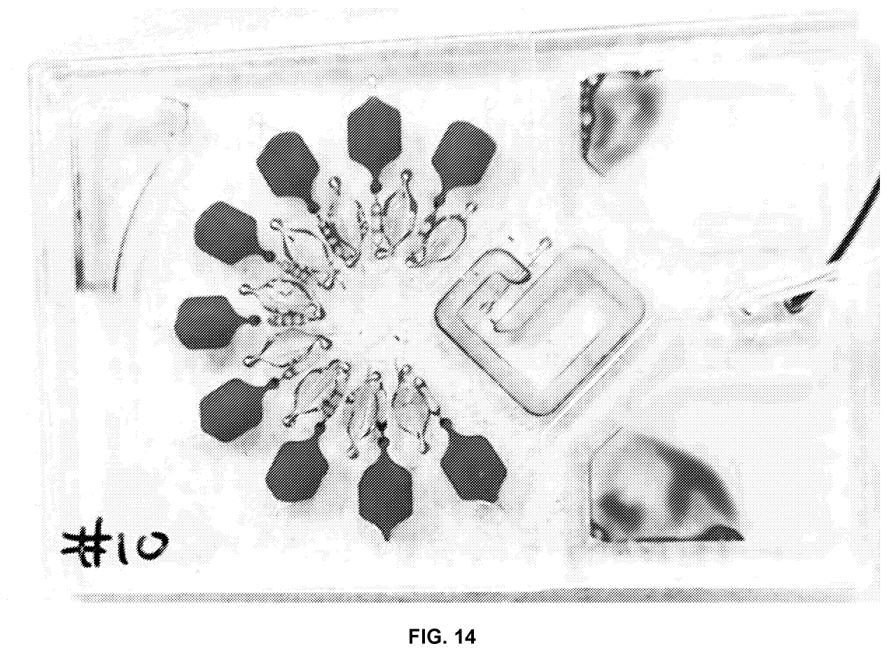

In various embodiments, particularly where the testing process involves multiple, sequential exposure conditions and/or may take longer than five minutes (e.g., up to sixty minutes) to complete, it may be desirable that the microchannels of a closed microfluidic cartridge facilitate some amount of fluid retention on their surface(s) (described herein as sacrificial fluid retention) to compensate for any evaporation of the sample within the cartridge during testing (see, e.g., FIG. 14, which shows fluid sample (which has been colored with a dark dye) along the interior walls of the microchannels even after the fluid sample aliquot has traveled all the way to the testing chambers). In some cases, it may be likely that, given the air (or other gas) that contacts the fluid sample (on the proximal or upstream side, and on the distal or downstream side), and the temperature in the cartridge, there may be evaporation of the fluid sample in each microchannel until the system reaches equilibrium. Evaporation of the sample can pose a problem in devices that use micro or nanodroplets for testing. Typically, these systems incorporate “sacrificial” droplets at specific points in the devices, the goal being that such “sacrificial” droplets, rather than the sample, evaporate and that the sample is therefore maintained in the liquid state at a constant volume. In certain embodiments of the present invention, as shown in FIG. 14, the materials that are used in assembling the cartridge components may be chosen or treated so that their surfaces that contact the fluid sample promote a certain amount of fluid sample retention along specific paths of each microfluidic channel (or at least those channels used for testing and comprising testing chambers). For example, if the exposed surface area of the fluid sample when it is in the testing chamber (the area of the fluid sample contacting air or other gas when the fluid sample is in the testing chamber) is smaller than the exposed surface area of the sacrificial fluid retention along the walls of the microchannel, the sacrificial fluid will be preferentially evaporated towards an equilibrated system, with minimal evaporation of the fluid sample and therefore minimal effect on the testing chamber sample volume. Channel surfaces may be coated (e.g., with a hydrophilic coating) to promote retention of sacrificial fluid. In certain embodiments, using a PSA layer to form the walls of the microfluidic channels can provide channel surfaces that promote sacrificial fluid retention; for example, for a PSA layer made of polycarbonate, the fluid sample tends to grab onto the PSA as it passes through the channel, resulting in the retention of sacrificial fluid. This sacrificial fluid is spread along the PSA lining the sides of the channels, giving the sacrificial fluid a larger surface area of exposure to the air (or other gas) in the cartridge compared to the exposed surface area of the sample in the testing chamber, as the sample in the testing chamber is exposed to the air (or other gas) only on two sides (the upstream and downstream sides, and the surface area of those sides further can be controlled with channel geometry).

While this invention has been particularly shown and described with references to certain embodiments thereof, it will be understood in light of the present disclosure by those skilled in the art that various changes in form and details may be made therein without departing from the scope of the invention, for example as encompassed by the appended claims.

What is claimed is:

1. A microfluidic cartridge comprising:
an inlet configured to receive a fluid sample, and
three or more channels that are connected at their upstream ends to the inlet, wherein:
at least two of said channels are testing channels, each testing channel comprising a metering chamber and a testing chamber, and
at least one of said channels is a waste channel comprising a waste chamber; and
wherein the microfluidic cartridge is a closed system such that air cannot vent out of said channels once a fluid sample is received at the inlet.

2. The microfluidic cartridge according to claim 1, wherein each testing channel further comprises a reagent chamber.

3. The microfluidic cartridge according to any one of claims 1-2, wherein the microfluidic cartridge further comprises two or more pairs of electrode sensors.

4. The microfluidic cartridge according to claim 3, wherein each pair of electrode sensors is positioned to measure an electrical property of the fluid sample.

5. The microfluidic cartridge according to any one of claims 1-2, wherein the microfluidic cartridge further comprises two or more fluidic position sensors.

6. The microfluidic cartridge according to claim 5, wherein each fluidic position sensor is a pair of electrode sensors.

7. The microfluidic cartridge according to any one of claims 5-6, wherein each fluidic position sensor is configured to detect the amount of fluid sample in one of the testing channels.

8. The microfluidic cartridge according to any one of claims 1-7, wherein the microfluidic cartridge comprises at least five testing channels.

9. A method of evaluating a fluid sample comprising inserting the fluid sample into the inlet of the microfluidic cartridge according to any one of claims 1-8.

10. The method according to claim 9, comprising splitting the fluid sample into the three or more channels that are connected at their upstream ends to the inlet, such that the fluid sample is split into three or more portions, with at least two of said portions being split into the testing channels and at least one of said portions being split into the waste channel.

11. The method according to claim 10, further comprising metering to a specific volume each portion that was split into one of the testing channels, wherein the metering is performed in the metering chamber of each testing channel.

12. The method according to claim 11, wherein all portions that were split into the testing channels are metered to the same volume.
